(19) United States
(12) Patent Application Publication
(10) Pub. No.: US 2025/0029419 A1
(43) Pub. Date: Jan. 23, 2025

(54) OPTICAL COHERENCE TOMOGRAPHY IMAGE GENERATION APPARATUS, OPTICAL COHERENCE TOMOGRAPHY IMAGE GENERATION METHOD, AND NON-TRANSITORY RECORDING MEDIUM

(71) Applicant: NEC Corporation, Minato-ku, Tokyo (JP)

(72) Inventors: John Kenji David CLARK, Tokyo (JP); Shigeru NAKAMURA, Tokyo (JP)

(73) Assignee: NEC Corporation, Minato-ku, Tokyo (JP)

(21) Appl. No.: 18/711,910

(22) PCT Filed: Mar. 25, 2022
(86) PCT No.: PCT/JP2022/014426

§ 371 Date: May 21, 2024

(51) Int. Cl. G06V 40/13; A61B 5/00; A61B 5/1172; G06T 7/73; G06V 40/19; G06V 40/50; G06V 40/60

(52) U.S. Cl. CPC G06V 40/1312; A61B 5/0066; A61B 5/1172; A61B 5/444; G06T 7/73; G06V 40/19; G06V 40/50; G06V 40/67; G06T 2200/04; G06T 2207/10012; G06T 2207/10101; G06T 2207/20021; G06T 2207/30041; G06T 2207/30088; G06T 2207/30201

(57) ABSTRACT

An optical coherence tomography image generation apparatus includes: an acquisition unit that acquires a stereoscopic 3D image of a target; a determination unit that determines a plurality of scan areas on the target, on the basis of the stereoscopic 3D image; and a control unit that relatively moves an irradiation position of light for capturing an optical coherence tomography image of the target with respect to the target, and that controls scanning by the light of each of the plurality of scan areas.

### TECHNICAL FIELD

This disclosure relates to technical fields of an optical coherence tomography image generation apparatus, an optical coherence tomography image generation method, and a recording medium.

### BACKGROUND ART

Patent Literature 1 discloses a technology/technique of: acquiring information on a specific area in biological pattern information that is detected on the basis the biological pattern information; performing control to display the biological pattern information by giving different display attributes to an area corresponding to the specific area and an area other than specific area, on the basis of the information on the specific area; and detecting the specific area even when a biological pattern has a specific area due to damage or the like, as well as presenting the detected specific area to a user in an easy-to-understand manner. Patent Literature 2 discloses a technology/technique of including: an observation system that captures an observation image of an anterior eye part of a subject eye with an imaging sensor via an objective lens; an interference optical system that has a branch optical path branching from the middle of an observation system optical path; an apparatus body that houses the observation system and the interference optical system; a relative movement mechanism that moves the apparatus body relative to the subject eye; an imaging control unit that controls the observation system and the interference optical system to cause the imaging sensor to capture the observation image and a part of return light while measurement light is applied to the subject eye from the interference optical system; and an XY alignment control unit that drives the relative movement mechanism automatically or manually on the basis of the observation image and the return light captured by the imaging sensor to perform XY alignment in an XY direction of the apparatus body with respect to a specific site of a cornea of the anterior eye part. Patent Literature 3 describes an ophthalmologic apparatus that is capable of preferably executing position matching between a subject eye and an apparatus optical system, the ophthalmologic apparatus including: an examination optical system for examining the subject eye; a supporting part configured to support a face of a subject; a driver configured to relatively and three-dimensionally move the examination optical system and the supporting part; two or more imaging parts configured to substantially simultaneously photograph an anterior eye part of the subject eye from different directions; an analyzer configured to obtain a three-dimensional position of the subject eye by analyzing two or more photograph images substantially simultaneously obtained by the two or more imaging parts; and a controller configured to relatively move the examination optical system and the supporting part by controlling the driver based on the obtained three-dimensional position. Patent Literature 4 describes a contactless finger print collation/matching apparatus that increases the accuracy of collation/matching by acquiring collation data that takes into account the attitude of a finger, the contactless finger print collation apparatus including: a camera unit and a laser irradiation unit for generating data on a finger face including a finger print; a measurement unit for measuring a three-dimensional position of the finger face on the basis of finger-face data; a calculation unit for computing a terminal axis direction on the basis of the measured three-dimensional position; a setting unit for setting a curvilinear coordinate system defining a curved surface formed by a first group of lines of intersection of the finger face and a longitudinal section group substantially parallel to the terminal axis direction and by a second group of lines of intersection of the finger face and a cross section group substantially perpendicular to the longitudinal section group; and a fingerprint image data acquisition unit for acquiring fingerprint image data represented in a predetermined plane coordinate system; and a collation data acquisition unit for producing, from the fingerprint image data, intermediate data represented in the curvilinear coordinate system and computing, from the intermediate data, collation data represented in a coordinate system of a virtual plane into which the curved surface corresponding to the curvilinear coordinate system is virtually developed.

### CITATION LIST

### Patent Literature

Patent Literature 1: JP2020-080188A
Patent Literature 2: JP2020-199210A
Patent Literature 3: JP2013-248376A
Patent Literature 4: JP2006-172258A

### SUMMARY

### Technical Problem

It is an example object of this disclosure to provide an optical coherence tomography image generation apparatus, an optical coherence tomography image generation method, and a recording medium that aim to improve the techniques/technologies disclosed in Citation List.

### Solution to Problem

An optical coherence tomography image generation apparatus according to an example aspect of this disclosure includes: an acquisition unit that acquires a stereoscopic 3D image of a target; a determination unit that determines a plurality of scan areas on the target, on the basis of the stereoscopic 3D image; and a control unit that relatively moves an irradiation position of light for capturing an optical coherence tomography image of the target with respect to the target, and that controls scanning by the light of each of the plurality of scan areas.

An optical coherence tomography image generation method according to an example aspect of this disclosure includes: acquiring a stereoscopic 3D image of a target; determining a plurality of scan areas on the target, on the basis of the stereoscopic 3D image; and relatively moving an irradiation position of light for capturing an optical coherence tomography image of the target with respect to the target, and controlling scanning by the light of each of the plurality of scan areas.

A recording medium according to an example aspect of this disclosure is a recording medium on which a computer program that allows a computer to execute an optical coherence tomography image generation method is recorded, the optical coherence tomography image generation method including: acquiring a stereoscopic 3D image of a target; determining a plurality of scan areas on the target, on the basis of the stereoscopic 3D image; and relatively moving an irradiation position of light for capturing an optical coherence tomography image of the target with respect to the target, and controlling scanning by the light of each of the plurality of scan areas.

### BRIEF DESCRIPTION OF DRAWINGS

FIG. 1 is a block diagram illustrating a configuration of an optical coherence tomography (OCT) image generation apparatus in a first example embodiment.

FIG. 2 is a block diagram illustrating a configuration of an optical coherence tomography image generation apparatus in a second example embodiment.

FIG. 3 is an external view illustrating the optical coherence tomography image generation apparatus in the second example embodiment.

FIG. 4 is a flowchart illustrating a flow of an optical coherence tomography image generation operation performed by the optical coherence tomography image generation apparatus in the second example embodiment.

FIG. 5 is a conceptual diagram illustrating the optical coherence tomography image generation operation performed by the optical coherence tomography image generation apparatus in the second example embodiment.

FIG. 6 illustrates a modified example of the optical coherence tomography image generation operation performed by the optical coherence tomography image generation apparatus in the second example embodiment.

FIG. 7 is a block diagram illustrating a configuration of an optical coherence tomography image generation apparatus in a third example embodiment.

FIG. 8 is a flowchart illustrating a flow of the optical coherence tomography image generation operation performed by the optical coherence tomography image generation apparatus in the third example embodiment.

FIG. 9 is a conceptual diagram illustrating the optical coherence tomography image generation operation performed by the optical coherence tomography image generation apparatus in the third example embodiment.

FIG. 10 illustrates a modified example of the optical coherence tomography image generation operation performed by the optical coherence tomography image generation apparatus in the third example embodiment.

FIG. 11 is a flowchart illustrating a flow of a scan area determination operation performed by an optical coherence tomography image generation apparatus in a fourth example embodiment.

FIG. 12 is a conceptual diagram illustrating the scan area determination operation performed by the optical coherence tomography image generation apparatus in the fourth example embodiment.

FIG. 13 is a flowchart illustrating a flow of the scan area determination operation performed by an optical coherence tomography image generation apparatus in a fifth example embodiment.

FIG. 14 is a block diagram illustrating a configuration of an optical coherence tomography image generation apparatus in a sixth example embodiment.

FIG. 15 is a flowchart illustrating a flow of the optical coherence tomography image generation operation performed by the optical coherence tomography image generation apparatus in the sixth example embodiment.

FIG. 16 is a block diagram illustrating a configuration of an optical coherence tomography image generation apparatus in a seventh example embodiment.

FIG. 17 is a flowchart illustrating a flow of the optical coherence tomography image generation operation performed by the optical coherence tomography image generation apparatus in the seventh example embodiment.

FIG. 18 is a conceptual diagram illustrating the optical coherence tomography image generation operation performed by the optical coherence tomography image generation apparatus in the seventh example embodiment.

FIG. 19 is a flowchart illustrating a flow of the optical coherence tomography image generation operation performed by an optical coherence tomography image generation apparatus in an eighth example embodiment.

FIG. 20 is a conceptual diagram illustrating the optical coherence tomography image generation operation performed by the optical coherence tomography image generation apparatus in the eighth example embodiment.

FIG. 21 is an external view illustrating an optical coherence tomography image generation apparatus in a ninth example embodiment.

FIG. 22 is an external view illustrating an optical coherence tomography image generation apparatus in a tenth example embodiment.

FIG. 23 is a block diagram illustrating a configuration of an optical coherence tomography image generation apparatus in an eleventh example embodiment.

FIG. 24 illustrates an example of a management screen displayed in the eleventh example embodiment.

FIG. 25 is a block diagram illustrating a configuration of an optical coherence tomography image generation apparatus in a twelfth example embodiment.

FIG. 26 is a flowchart illustrating a flow of the optical coherence tomography image generation operation performed by the optical coherence tomography image generation apparatus in the twelfth example embodiment.

### DESCRIPTION OF EXAMPLE EMBODIMENTS

Hereinafter, an optical coherence tomography image generation apparatus, an optical coherence tomography image generation method, and a recording medium according to example embodiments will be described with reference to the drawings.

### 1: FIRST EXAMPLE EMBODIMENT

An optical coherence tomography image generation apparatus, an optical coherence tomography image generation method, and a recording medium according to a first example embodiment will be described. The following describes the optical coherence tomography image generation apparatus, the optical coherence tomography image generation method, and the recording medium according to the first example embodiment, by using an optical coherence tomography image generation apparatus 1 to which the optical coherence tomography image generation apparatus, the optical coherence tomography image generation method, and the recording medium according to the first example embodiment are applied.

### [1-1: Configuration of Optical Coherence Tomography Image Generation Apparatus 1]

With reference to FIG. 1, a configuration of the optical coherence tomography image generation apparatus 1 in the first example embodiment will be described. FIG. 1 is a block diagram illustrating the configuration of the optical coherence tomography image generation apparatus 1 in the first example embodiment.

As illustrated in FIG. 1, the optical coherence tomography image generation apparatus 1 includes an acquisition unit 11, a determination unit 12, and a control unit 13. The acquisition unit 11 acquires a stereoscopic three-dimensional (3D) image SI of a target. The determination unit 12 determines a plurality of scan areas on the target, on the basis of the stereoscopic 3D image SI. The control unit 13 relatively moves an irradiation position of light for capturing an optical coherence tomography image of the target with respect to the target, and controls scanning by the light of each of the plurality of scan areas.

### [1-2: Technical Effect of Optical Coherence Tomography Image Generation Apparatus 1]

The optical coherence tomography image generation apparatus 1 in the first example embodiment is capable of easily accurately determining the plurality of scan areas and generating the optical coherence tomography image, accurately, by using the stereoscopic 3D image SI.

### 2: SECOND EXAMPLE EMBODIMENT

An optical coherence tomography image generation apparatus, an optical coherence tomography image generation method, and a recording medium according to a second example embodiment will be described. The following describes the optical coherence tomography image generation apparatus, the optical coherence tomography image generation method, and the recording medium according to the second example embodiment, by using an optical coherence tomography image generation apparatus 2 to which the optical coherence tomography image generation apparatus, the optical coherence tomography image generation method, and the recording medium according to the second example embodiment are applied.

### [2-1: Configuration of Optical Coherence Tomography Image Generation Apparatus 2]

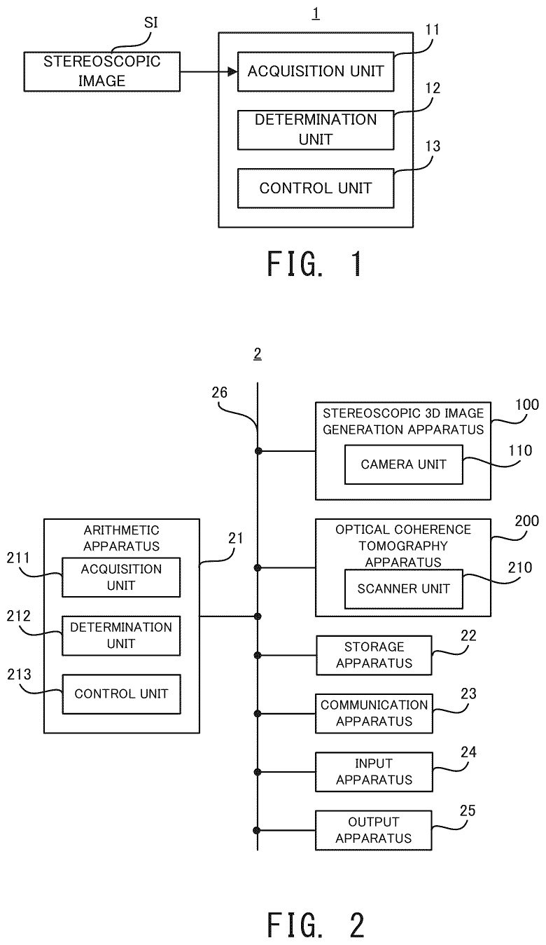

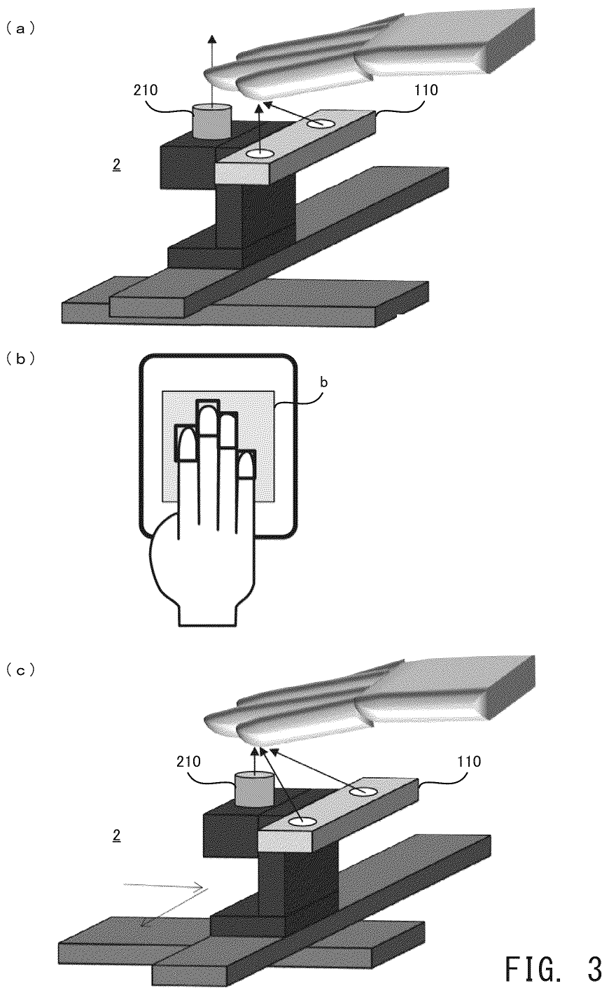

With reference to FIG. 2, a configuration of the optical coherence tomography image generation apparatus 2 in the second example embodiment will be described. FIG. 2 is a block diagram illustrating the configuration of the optical coherence tomography image generation apparatus 2 in the second example embodiment.

As illustrated in FIG. 2, the optical coherence tomography image generation apparatus 2 includes an arithmetic apparatus 21 and a storage apparatus 22. Furthermore, the optical coherence tomography image generation apparatus 2 may include a stereoscopic 3D image generation apparatus 100, an optical coherence tomography apparatus 200, a communication apparatus 23, an input apparatus 24, and an output apparatus 25. The optical coherence tomography image generation apparatus 2, however, may not include at least one of the stereoscopic 3D image generation apparatus 100, the optical coherence tomography apparatus 200, the communication apparatus 23, the input apparatus 24, and the output apparatus 25. In a case where the optical coherence tomography image generation apparatus 2 does not include at least one of the stereoscopic 3D image generation apparatus 100 and the optical coherence tomography apparatus 200, the optical coherence tomography image generation apparatus 2 may perform transmission and reception of information through the communication apparatus 23, with the optical coherence tomography apparatus 200 and the stereoscopic 3D image generation apparatus 100. The arithmetic apparatus 21, the storage apparatus 22, the stereoscopic 3D image generation apparatus 100, the optical coherence tomography apparatus 200, the communication apparatus 23, the input apparatus 24 and the output apparatus 25 may be connected through a data bus 26.

The arithmetic apparatus 21 includes at least one of a CPU (Central Processing Unit), a GPU (Graphics Processing Unit), and a FPGA (Field Programmable Gate Array), for example. The arithmetic apparatus 21 reads a computer program. For example, the arithmetic apparatus 21 may read a computer program stored in the storage apparatus 22. For example, the arithmetic apparatus 21 may read a computer program stored by a computer-readable and non-transitory recording medium, by using a not-illustrated recording medium reading apparatus provided in the optical coherence tomography image generation apparatus 2 (e.g., the input apparatus 24 described later). The arithmetic apparatus 21 may acquire (i.e., download or read) a computer program from a not-illustrated apparatus disposed outside the optical coherence tomography image generation apparatus 2, through the communication apparatus 23 (or another communication apparatus). The arithmetic apparatus 21 executes the read computer program. Consequently, a logical functional block for performing an operation to be performed by the optical coherence tomography image generation apparatus 2 is realized or implemented in the arithmetic apparatus 21. That is, the arithmetic apparatus 21 is allowed to function as a controller for realizing or implementing the logical functional block for performing an operation (in other words, a processing) to be performed by the optical coherence tomography image generation apparatus 2.

FIG. 2 illustrates an example of the logical functional block realized or implemented in the arithmetic apparatus 21 to perform an optical coherence tomography image generation operation. As illustrated in FIG. 2, an acquisition unit 211 that is a specific example of the “acquisition unit”, a determination unit 212 that is a specific example of the “determination unit”, and a control unit 213 that is a specific example of the “control unit” are realized or implemented in the arithmetic apparatus 21. Each operation of the acquisition unit 211, the determination unit 212, and the control unit 213 will be described later with reference to FIG. 3 to FIG. 5.

The storage apparatus 22 is configured to store desired data. For example, the storage apparatus 22 may temporarily store a computer program to be executed by the arithmetic apparatus 21. The storage apparatus 22 may temporarily store data that are temporarily used by the arithmetic apparatus 21 when the arithmetic apparatus 21 executes the computer program. The storage apparatus 22 may store data that are stored by the optical coherence tomography image generation apparatus 2 for a long time. The storage apparatus 22 may include at least one of a RAM (Random Access Memory), a ROM (Read Only Memory), a hard disk apparatus, a magneto-optical disk apparatus, a SSD (Solid State Drive), and a disk array apparatus. That is, the storage apparatus 22 may include a non-transitory recording medium.

The communication apparatus 23 is configured to communicate with an apparatus external to the optical coherence tomography image generation apparatus 2 through a not-illustrated communication network. The communication apparatus 23 may be a communication interface based on a standard such as Ethernet, Wi-Fi, Bluetooth, and USB (Universal Serial Bus. In a case where the communication apparatus 23 is a communication interface based on the USB standard, the communication apparatus 23 may be capable of communicate, for example, with the arithmetic apparatus 21 including a FPGA and a mechanism including a computer for controlling the entire optical coherence tomography image generation apparatus 2.

The input apparatus 24 is an apparatus that receives an input of information to the optical coherence tomography image generation apparatus 2 from an outside of the optical coherence tomography image generation apparatus 2. For example, the input apparatus 24 may include an operating apparatus (e.g., at least one of a keyboard, a mouse trackball, a touch panel, a pointing device such as a pen tablet, a button, and the like) that is operable by an operator of the optical coherence tomography image generation apparatus 2. For example, the input apparatus 24 may include a reading apparatus that is configured to read information recorded as data on a recording medium that is externally attachable to the optical coherence tomography image generation apparatus 2.

The output apparatus 25 is an apparatus that outputs information to the outside of the optical coherence tomography image generation apparatus 2. For example, the output apparatus 25 may output information as an image. That is, the output apparatus 25 may include a display apparatus (a so-called display) that is configured to display an image indicating the information that is desirably outputted. Examples of the display apparatus include a liquid crystal display, an OLED (Organic Light Emitting Diode) display, and the like. For example, the output apparatus 25 may output information as audio/sound. That is, the output apparatus 25 may include an audio apparatus (a so-called speaker) that is configured to output audio/sound. For example, the output apparatus 25 may output information onto a paper surface. That is, the output apparatus 25 may include a print apparatus (a so-called printer) that is configured to print desired information on the paper surface. The input apparatus 24 and the output apparatus 25 may be integrally formed as a touch panel.

The hardware configuration illustrated in FIG. 2 is an example. An apparatus other than apparatuses illustrated in FIG. 2 may be added, and a part of the apparatuses may not be provided. In addition, a part of the apparatuses may be substituted by another apparatus having a same/similar function. In addition, a part of the functions in the second example embodiment may be provided by another apparatus through a network. The functions in the second example embodiment may be distributed to and realized in a plurality of apparatuses. As described above, the hardware configuration illustrated in FIG. 2 may be changed as appropriate.

[2-2: Stereoscopic 3D image Generation Apparatus 100]

The stereoscopic 3D image generation apparatus 100 generates the stereoscopic 3D image SI of the target. The stereoscopic 3D image generation apparatus 100 may be a stereo camera. The stereoscopic 3D image generation apparatus 100 may include at least two camera units 110 that are at different positions with respect to the target. The stereoscopic 3D image generation apparatus 100 may include at least two camera units 110 with different imaging angles to the target. The stereoscopic 3D image generation apparatus 100 may generate the stereoscopic 3D image SI from a plurality of the target images captured from different angles. The stereoscopic 3D image SI generated by the stereoscopic 3D image generation apparatus 100 may be used to acquire a three-dimensional position of an area of the target on which optical coherence tomography scanning (OCT scanning) is to be performed. The stereoscopic 3D image generation apparatus 100 may generate the stereoscopic 3D image SI that allows acquisition of the three-dimensional position of each part of the target.

The operation of generating the stereoscopic 3D image SI of the stereoscopic 3D image generation apparatus 100 may be controlled by the control unit 213. The control unit 213 may perform a movement control and an imaging control of the camera unit 110.

### [2-3: Optical Coherence Tomography Apparatus 200]

The optical coherence tomography apparatus 200 irradiates the target with a light beam while performing two-dimensional scanning on the target, performs the optical coherence tomography, and generates three-dimensional luminance data on the target.

The optical coherence tomography is a technique/technology of identifying a position in an optical axis direction of a light scattering point where object light is scattered in the target, i.e., in a depth direction of the target, by using interference between the object light and reference light, and acquiring structural data spatially resolved in the depth direction of an inside of the target. The optical coherence tomography technique/technology includes Time Domain (TD-OCT) and

Fourier Domain (FD-OCT), but the FD-OCT is adopted in the second example embodiment. In the FD-OCT, an interference light spectrum in a wide wavelength band is measured in the interference of the object light and the reference light, and is Fourier-transformed to acquire the structural data in the depth direction. A method of acquiring the interference light spectrum includes Spectral Domain (SD-OCT) using a spectrometer and Swept Source (SS-OCT) using a light source for sweeping a wavelength. The optical coherence tomography image generation apparatus 2 in the second example embodiment performs the OCT scanning in the SS-OCT.

The optical coherence tomography apparatus 200 may scan an irradiation position of the object light in an in-plane direction perpendicular to the depth direction of the target, thereby to acquire tomography structural data spatially resolved in the in-plane direction and structural data spatially resolved in the depth direction, i.e., three-dimensional tomography structural data on a measuring the target object.

The optical coherence tomography apparatus 200 may include a light source, a scanner unit 210, and a signal processing unit. An optical coherence tomography operation of the optical coherence tomography apparatus 200 may be controlled by the control unit 213. The control unit 213 may control movement, a scanning position, and a scanning velocity of the scanner unit 210.

The light source may emit light while sweeping the wavelength. The scanner unit 210 irradiates the target with the object light emitted from the light source and scatters the object light. The object light scattered from the target interferes with the reference light reflected by a reference light mirror, and two interference lights are generated. That is, an intensity ratio of the two interference lights is determined by a phase difference between the object light and the reference light. The scanner unit 210 outputs an electrical signal corresponding to the intensity difference of the two interference lights to the signal processing unit.

The signal processing unit digitizes and processes the electrical signal outputted by the scanner unit 210. The signal processing unit performs Fourier transform on generated interference light spectrum data, and acquires data indicating the intensity of backscattered light (object light) at different depth positions in the depth direction (also referred to as a “Z direction”). The operation of acquiring the data indicating the intensity of the backscattered light (object light) in the depth direction (Z direction) of the irradiation position of the object light in the target is referred to as “A-scan”. The signal processing unit generates a waveform indicating object light backscatter intensity at an Nz point, as an A-scan waveform.

The scanner unit 210 scans the irradiation position of the object light on the target. The scanner unit 210 moves the irradiation position of the object light in a scanning line direction (also referred to as a “fast axis direction of the scanning” and an “X direction”).

The signal processing unit repeats the A-scan operation at each irradiation position of the object light, and connects the A-scan waveforms at the respective irradiation positions of the object light. As a result, the signal processing unit acquires a map of the intensity of the two-dimensional backscattered light (object light) in the scanning line direction (X direction) and in the depth direction (Z direction), as a tomography image. Hereinafter, an operation of repeating the A-scan operation while moving in the scanning line direction (the fast axis direction of the scanning, the X direction) and connecting measurement results is referred to as “B-scan”. In a case where the irradiation position of the object light for each B-scan is an Nx position, the tomography image by the B-scan is two-dimensional luminance data indicating the object light backscatter intensity at Nz×Nx points.

The scanner unit 210 moves the irradiation position of the object light not only in the scanning line direction (X direction), but also in a direction perpendicular to the scanning line (also referred to as a “slow axis direction of the scanning” and a “Y direction”). The signal processing unit repeats the B-scan operation and connects B-scan measurement results. In this way, the signal processing unit acquires three-dimensional tomography structural data. Hereinafter, an operation of repeating the B scan operation while moving in the direction perpendicular to the scanning line (Y direction) and connecting measurement results is referred to as “C scan”. In a case where the number of the B-scans performed for each C-scan is Ny, the tomography structural data acquired by the C-scan are three-dimensional luminance data indicating the object light backscatter intensity at Nz×Nx×Ny points.

The signal processing unit transmits digitized data to the arithmetic apparatus 21. The operation by the signal processing unit may be performed by the arithmetic apparatus 21.

FIG. 3(a) is an external view illustrating the optical coherence tomography image generation apparatus 2 in the second example embodiment. The scanner unit 210 and the camera unit 110 may be fixed to the same stage and integrated, as illustrated in FIG. 3(a).

The optical coherence tomography image generation apparatus 2 may image fingers of a hand. The optical coherence tomography image generation apparatus 2 maty be configured, as illustrated in FIG. 3(b), such that a palm is directed downward to hold the fingers over the camera unit 110 of the stereoscopic 3D image generation apparatus 100 and the scanner unit 210 of the optical coherence tomography apparatus 200. FIG. 3(b) illustrates an imaging area b of the stereoscopic 3D image generation apparatus 100. In the example illustrated in FIG. 3(b), the stereoscopic 3D image generation apparatus 100 may capture the stereoscopic 3D images SI of the second to fourth fingers of one hand. Alternatively, the optical coherence tomography image generation apparatus 2 may be configured such that the hand is placed on a placing table with the palm facing up, thereby to image the fingers of the hand from the top.

The control unit 213 may move the scanner unit 210 in accordance with the scan areas determined on the basis of the stereoscopic 3D image SI. The scanner unit 210 and the camera unit 110 may be fixed to the same stage and integrally moved, as illustrated in FIG. 3(c). Alternatively, the scanner unit 210 and the camera section 110 may be separately moved.

By the way, there is an upper limit on a size of an area in which the three-dimensional luminance data can be acquired by one C-scan. For example, the size of the area in which the three-dimensional luminance data can be acquired by the C-scan is much smaller than a size of an area that can be included in the stereoscopic 3D image SI by one-time generation of the stereoscopic 3D image SI. In contrast, by determining in advance a three-dimensional position of a desired area in which the three-dimensional luminance data are desirably acquired, it is possible to efficiently acquire the high-accuracy, three-dimensional luminance data on the desired area. Therefore, the optical coherence tomography image generation apparatus 2 in the second example embodiment determines the plurality of scan areas on the target area, on the basis of the stereoscopic 3D image SI, before the generation of the optical coherence tomography image.

### [2-4: Optical Coherence Tomography Image Generation Operation Performed by Optical Coherence Tomography Image Generation Apparatus 2]

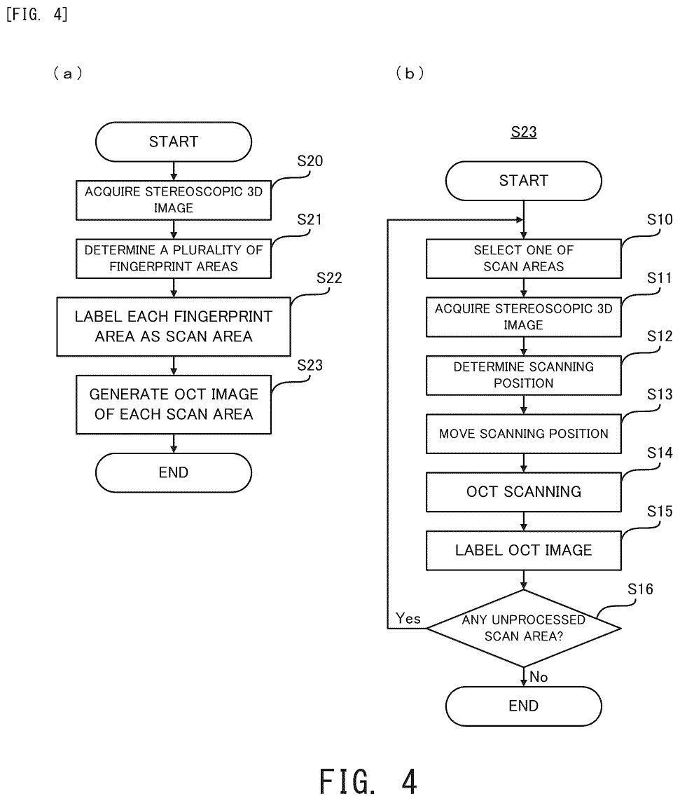

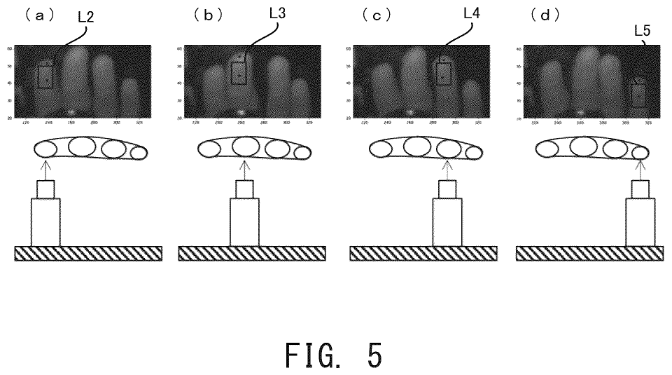

With reference to FIG. 4 and FIG. 5, a flow of the optical coherence tomography image generation operation performed by the optical coherence tomography apparatus 2 in the second example embodiment will be described. FIG. 4 is a flowchart illustrating the flow of the optical coherence tomography image generation operation performed by the optical coherence tomography image generation apparatus 2 in the second example embodiment. FIG. 5 is a conceptual diagram illustrating the optical coherence tomography image generation operation performed by the optical coherence tomography image generation apparatus 2 in the second example embodiment.

In the second example embodiment, the target of the optical coherence tomography may be a hand. In the second example embodiment, the determination unit 212 may determine respective fingerprint areas of two or more of the fingers of the hand, as the plurality of scan areas, on the basis of the stereoscopic 3D image SI.

As illustrated in FIG. 4(a), the acquisition unit 211 acquires the stereoscopic 3D image SI of the hand serving as the target (step S20). The acquisition unit 211 may acquire the stereoscopic 3D image SI of the hand generated by the stereoscopic 3D image generation apparatus 100.

The determination unit 212 determines the respective fingerprint areas of two or more of the fingers of the hand, as the plurality of scan areas, on the basis of the stereoscopic 3D image SI of the hand (step S21). The determination unit 212 may estimate fingertips of two or more of the fingers of the hand, on the basis of the stereoscopic 3D image SI of the hand, and may determine the fingerprint area including at least a part of an area extending to a first joint on the finger from the fingertip toward a base of the finger, as at least one of the plurality of scan areas. As illustrated in FIG. 5, the determination unit 212 may determine each of (a) a fingerprint area L2 of the second finger, (b) a third fingerprint area L3 of the third finger, (c) a fourth fingerprint area L4 of the fourth finger, and (d) a fifth fingerprint area L5 of the fifth finger, of a left hand, as the plurality of scan areas. For example, as illustrated in FIG. 5, the determination unit 212 may determine a rectangular area of each finger, as the fingerprint area.

The determination unit 212 labels each of the plurality of fingerprint areas (step S22). For example, as illustrated in FIG. 5, the determination unit 212 may label (a) the second fingerprint area of the left hand as “L2.” In addition, the determination unit 212 may label (b) the third fingerprint area of the left hand as “L3.” The determination unit 212 may label (c) the fourth fingerprint area of the left hand as “L4.” In addition, the determination unit 212 may label (d) the fifth fingerprint area of the left hand as “L5.”

The control unit 213 generates the optical coherence tomography image of each scan area (step S23). The operation of the step S23 is illustrated in FIG. 4(b). As illustrated in FIG. 4(b), the control unit 213 selects one of the plurality of fingerprint areas (step S10). For example, the determination unit 212 may firstly select the fingerprint area L2 of the second finger of the left hand.

The acquisition unit 211 acquires the stereoscopic 3D image SI of the selected one fingerprint area (step S11). The acquisition unit 211 may acquire the stereoscopic 3D image SI of one fingerprint area generated by the stereoscopic 3D image generation apparatus 100. The control unit 213, however, may not acquire the stereoscopic 3D image SI of the selected one fingerprint area. Since the operation of acquiring the stereoscopic 3D image SI of the fingerprint area in the step S11 is a processing for a case where the hand moves, the operation of acquiring the stereoscopic 3D image SI of the fingerprint area may be omitted for the firstly selected one fingerprint area, for example.

The determination unit 212 determines an optical coherence tomography scanning position corresponding to the selected one fingerprint area, on the basis of the stereoscopic 3D image SI (step S12).

The control unit 213 moves the scanner unit 210 to the optical coherence tomography scanning position corresponding to the one fingerprint area (step S13). For example, as illustrated in a lower part of FIG. 5, the control unit 213 may move the scanner unit 210 to the optical coherence tomography scanning position corresponding to selected one of the fingerprint areas L2, L3, L4, and L5.

The control unit 213 relatively moves the irradiation position of light for capturing the optical coherence tomography image of the one fingerprint area with respect to the one fingerprint area, and controls scanning by the light of the one fingerprint area (step S14). The control unit 213 may control the optical coherence tomography scanning by the scanner unit 210.

The determination unit 212 performs the same labeling as that of the fingerprint area, on the captured optical coherence tomography image of the one fingerprint area (step S15).

The determination unit 212 determines whether or not there is a fingerprint area in which the step S10 to the step S15 are not yet performed (step S16). When there is a fingerprint area in which the step S10 to the step S15 are not yet performed (the step S16: Yes), the operation proceeds to the step S10. In the step S10, the determination unit 212 may then select the fingerprint area L3 of the third finger of the left hand. Furthermore, the determination unit 212 may then select the fingerprint area L4 of the fourth finger of the left hand. Lastly, the determination unit 212 may select the fingerprint area L5 of the fifth finger of the left hand.

When there is no fingerprint area in which the step S10 to the step S15 are not yet performed (the step S16: No), the optical coherence tomography image generation operation performed by the optical coherence tomography apparatus 2 in the second example embodiment is ended. The determination unit 212 determines how many fingers appear, from the stereoscopic 3D image SI, and may repeat the step S10 to the step S15 as many times as the number of the fingers.

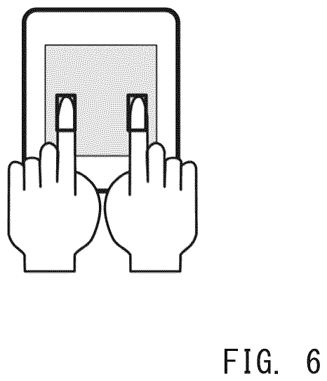

With reference to the FIG. 5, a description is made to the case where the optical coherence tomography image generation apparatus 2 generates the optical coherence tomography images of the second to fourth fingers of one hand, but the generation is not limited to that of the optical coherence tomography images of the fingers of one hand. For example, as illustrated in FIG. 6, the optical coherence tomography image generation apparatus 2 may generate the optical coherence tomography image of the fingers of both hands.

### [2-5: Technical Effect of Optical Coherence Tomography Image Generation Apparatus 2]

The optical coherence tomography image generation apparatus 2 in the second example embodiment is capable of generating the optical coherence tomography images of a plurality of points. The size of the optical coherence tomography image that can be acquired by the one-time optical coherence tomography operation is determined; however, the optical coherence tomography image generation apparatus 2 is capable of easily and accurately determining the fingerprint areas of the fingers of the hand by using the stereoscopic 3D image SI, thereby to generate a desired optical coherence tomography image.

### 3: THIRD EXAMPLE EMBODIMENT

An optical coherence tomography image generation apparatus, an optical coherence tomography image generation method, and a recording medium according to a third example embodiment will be described. The following describes the optical coherence tomography image generation apparatus, the optical coherence tomography image generation method, and the recording medium according to the third example embodiment, by using an optical coherence tomography image generation apparatus 3 to which the optical coherence tomography image generation apparatus, the optical coherence tomography image generation method, and the recording medium according to the third example embodiment are applied.

### [3-1: Configuration of Optical Coherence Tomography Image Generation Apparatus 3]

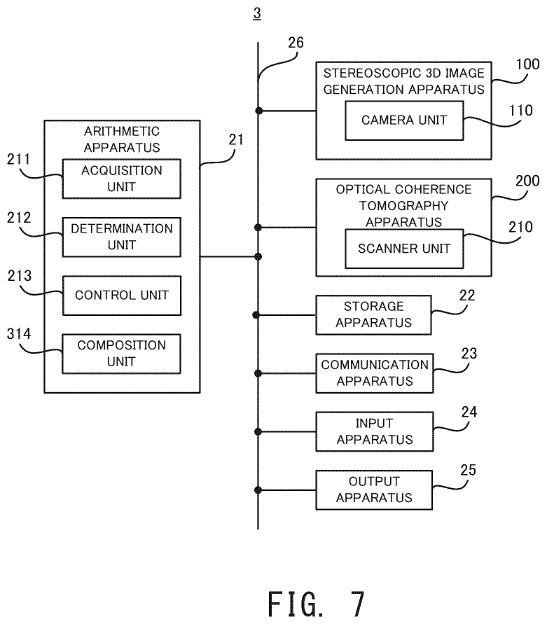

With reference to FIG. 7, a configuration of the optical coherence tomography image generation apparatus 3 in the third example embodiment will be described. FIG. 7 is a block diagram illustrating the configuration of the optical coherence tomography image generation apparatus 3 in the third example embodiment.

As illustrated in FIG. 7, the optical coherence tomography image generation apparatus 3 in the third example embodiment includes the arithmetic apparatus 21 and the storage apparatus 22, as in the optical coherence tomography image generation apparatus 2 in the second example embodiment. Furthermore, the optical coherence tomography image generation apparatus 3 may include the communication apparatus 23, the input apparatus 24, and the output apparatus 25, as in the optical coherence tomography image generation apparatus 2 in the second example embodiment. The optical coherence tomography image generation apparatus 3, however, may not include at least one of the communication apparatus 23, the input apparatus 24, and the output apparatus 25. The optical coherence tomography image generation apparatus 3 in the third example embodiment is different from the optical coherence tomography image generation apparatus 2 in the second example embodiment, in the determination operation by the determination unit 212 and in that the determination unit 212 provided in the arithmetic apparatus 21 includes a composition unit 314. The composition unit 314 generates the optical coherence tomography image of the desired area, on the basis of the optical coherence tomography image of each scan area. Other features of the optical coherence tomography image generation apparatus 3 may be the same as those of the optical coherence tomography image generation apparatus 2 in the second example embodiment.

### [3-2: Optical Coherence Tomography Image Generation Operation Performed by Optical Coherence Tomography Image Generation Apparatus 3]

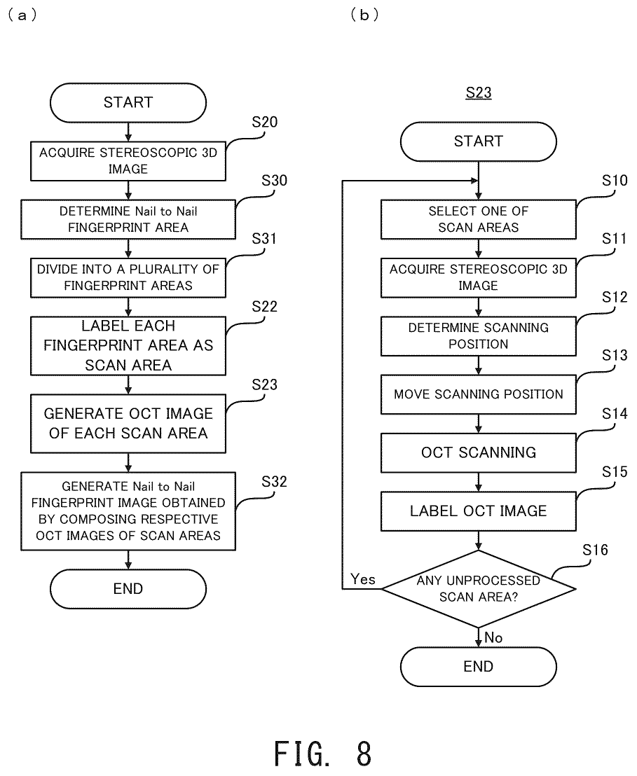

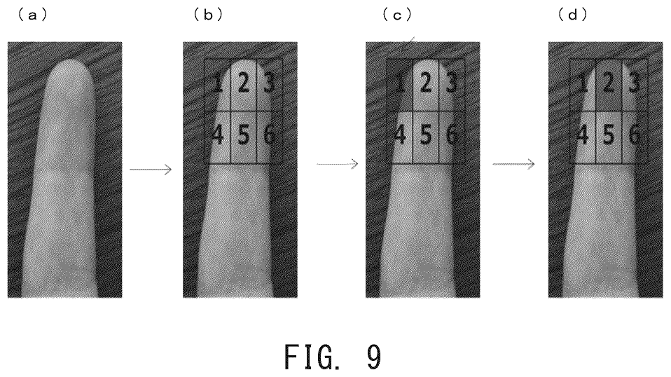

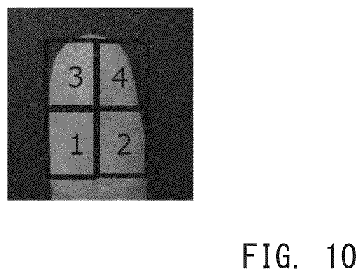

With reference to FIG. 8 and FIG. 9, a flow of the optical coherence tomography image generation operation performed by the optical coherence tomography apparatus 3 in the third example embodiment will be described. FIG. 8 is a flowchart illustrating the flow of the optical coherence tomography image generation operation performed by the optical coherence tomography image generation apparatus 3 in the third example embodiment. FIG. 9 is a conceptual diagram illustrating the optical coherence tomography image generation operation performed by the optical coherence tomography image generation apparatus 3 in the third example embodiment.

In the third example embodiment, the target of the optical coherence tomography image generation may be a hand. In the third example embodiment, the determination unit 212 determines an imaging area on the target on the basis of the stereoscopic 3D image SI, and divides the imaging area to determine the plurality of scan areas.

As illustrated in FIG. 8(a), the acquisition unit 211 acquires the stereoscopic 3D image SI of the hand serving as the target (step S20). For example, as illustrated in FIG. 9(a), the acquisition unit 211 may acquire the stereoscopic 3D image SI of one of the fingers of the hand.

The determination unit 212 determines a Nail to Nail fingerprint area serving as the imaging area on the finger, on the basis of the stereoscopic 3D image SI (step S30). For example, as illustrated in FIG. 9(b), the determination unit 212 may determine a rectangular area including a fingerprint of an entire area from the fingertip to the first joint, as the Nail to Nail fingerprint area. The determination unit 212 may estimate the fingertip of at least one of the fingers of the hand, on the basis of the stereoscopic 3D image SI of the hand, and may determine the fingerprint area including at least a part of an area extending to the first joint on the finger from the fingertip toward the base of the finger. The determination unit 212 may determine the fingerprint area of a larger size than an image size acquired by the optical coherence tomography scanning at a time, on the basis of the stereoscopic 3D image SI of the hand.

The determination unit 212 divides the Nail to Nail fingerprint area, and determines a plurality of fingerprint areas serving as the plurality of scan areas (step S31). For example, as illustrated in FIG. 9(b), the determination unit 212 may divides the Nail to Nail fingerprint area into six pieces, thereby to determine six fingerprint areas.

The determination unit 212 labels each of the plurality of fingerprint areas (step S22). For example, as illustrated in FIG. 9(b), the determination unit 212 may label an upper left fingerprint area of the Nail to Nail fingerprint area as “1.” In addition, the determination unit 212 may label an upper middle fingerprint area of the Nail to Nail fingerprint area as “2.” In addition, the determination unit 212 may label an upper right fingerprint area of the Nail to Nail fingerprint area as “3.” In addition, the determination unit 212 may label a lower left fingerprint area of the Nail to Nail fingerprint area as “4.” In addition, the determination unit 212 may label a lower middle fingerprint area of the Nail to Nail fingerprint area as “5.” In addition, the determination unit 212 may label a lower right fingerprint area of the Nail to Nail fingerprint area as “6.”

The control unit 213 generates the optical coherence tomography image of each scan area (step S23). The operation of the step S23 is illustrated in FIG. 8(b). As illustrated in FIG. 8(b), the control unit 213 selects one of the plurality of fingerprint areas (step S10). For example, the determination unit 212 may firstly select the fingerprint area L2 of the second finger of the left hand. For example, as illustrated in FIG. 9(c), the determination unit 212 may firstly select the upper left area 1.

The acquisition unit 211 acquires the stereoscopic 3D image SI of the selected one fingerprint area (step S11). A in the second example embodiment, however, the control unit 213 may not acquire the stereoscopic 3D image SI of the selected one fingerprint area.

The determination unit 212 determines the OCT scanning position corresponding to the selected one fingerprint area, on the basis of the stereoscopic 3D image SI (step S12).

The control unit 213 moves the scanner unit 210 to the OCT scanning position corresponding to the one fingerprint area (step S13). For example, as illustrated in FIG. 9(c), the control unit 213 may move the scanner unit 210 to the OCT scanning position corresponding to the firstly selected upper left area 1.

The control unit 213 relatively moves the irradiation position of the light for capturing the optical coherence tomography image of the one fingerprint area with respect to the one fingerprint area, and controls the scanning by light of the one fingerprint area (step S14). The control unit 213 may control the OCT scan by the scanner unit 210.

The determination unit 212 performs the same labeling as that of the fingerprint area, on the captured optical coherence tomography image of the one fingerprint area (step S15).

The determination unit 212 determines whether or not there is a fingerprint area in which the step S10 to the step S15 are not yet performed (step S16). When there is a fingerprint area in which the step S10 to the step S15 are not yet performed (the step S16: Yes), the operation proceeds to the step S10. In the step S10, for example, as illustrated in FIG. 9(d), the determination unit 212 may then select the upper middle area 2. Furthermore, the determination unit 212 may select the upper right area 3, the lower left area 4, the lower middle area 5, and the lower right area 6 in order.

When there is no fingerprint area in which the step S10 to the step S15 are not yet performed (the step S16: No), the operation proceeds to a step S32. The determination unit 212 may repeat the step S10 to the step S15 as many times as the number of divisions of the Nail to Nail fingerprint area.

The composition unit 314 generates a Nail to Nail fingerprint image obtained by composing the respective optical coherence tomography images of the fingerprint areas (step S32).

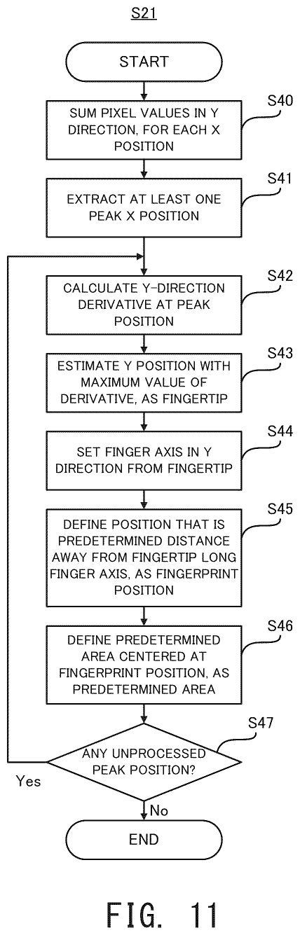

With reference to FIG. 9, a description is made to the case where the determination unit 212 divides the Nail to Nail area into six pieces, thereby to determine six fingerprint areas, but the number of divisions is not limited to six. For example, as illustrated in FIG. 10, the determination unit 212 may divide the Nail to Nail area into four pieces, thereby to determine four fingerprint areas. The determination unit 212 may divide the Nail to Nail area into an arbitrary number of pieces in accordance with a desired size of the optical coherence tomography image, thereby to determine an arbitrary numbers fingerprint areas.

Furthermore, the optical coherence tomography image generation apparatus 3 in the third example embodiment captures the optical coherence tomography image of a fingerprint image of one of the fingers of the hand, but may capture the optical coherence tomography images of more than one of the fingers of the hand. For example, the optical coherence tomography image generation apparatus 3 may capture the optical coherence tomography images of all of the first to fifth fingers. In this case, for example, the determination unit 212 may not divide the fingerprint areas of the second to fifth fingers, but divide the fingerprint of the first finger, thereby to determine a plurality of fingerprint areas.

In the third example embodiment, described is the case where the target is a hand as an example, but the target is not limited to the hand. The optical coherence tomography image generation apparatus 3 in the third example embodiment is applicable to a target other than hand, as described in other example embodiments described later.

### [3-3: Technical Effect of Optical Coherence Tomography Image Generation Apparatus 3]

The optical coherence tomography image generation apparatus 3 in the third example embodiment is capable of acquiring the optical coherence tomography image of the desired area, even in a case where the desired area in which the optical coherence tomography image is desirably acquired is greater than the area acquired by one-time optical coherence tomography.

### 4: FOURTH EXAMPLE EMBODIMENT

An optical coherence tomography image generation apparatus, an optical coherence tomography image generation method, and a recording medium according to a fourth example embodiment will be described. The following describes the optical coherence tomography image generation apparatus, the optical coherence tomography image generation method, and the recording medium according to the fourth example embodiment, by using an optical coherence tomography image generation apparatus 4 to which the optical coherence tomography image generation apparatus, the optical coherence tomography image generation method, and the recording medium according to the fourth example embodiment are applied.

The optical coherence tomography image generation apparatus 4 in the fourth example embodiment is different in the determination operation by the determination unit 212, from the optical coherence tomography image generation apparatus 2 in the second example embodiment and the optical coherence tomography image generation apparatus 3 in the third example embodiment. Other features of the optical coherence tomography image generation apparatus 4 may be the same as those of at least one of the optical coherence tomography image generation apparatus 2 and the optical coherence tomography apparatus 3.

### [4-1: Fingerprint Area Determination Operation Performed by Optical Coherence Tomography Image Generation Apparatus 4]

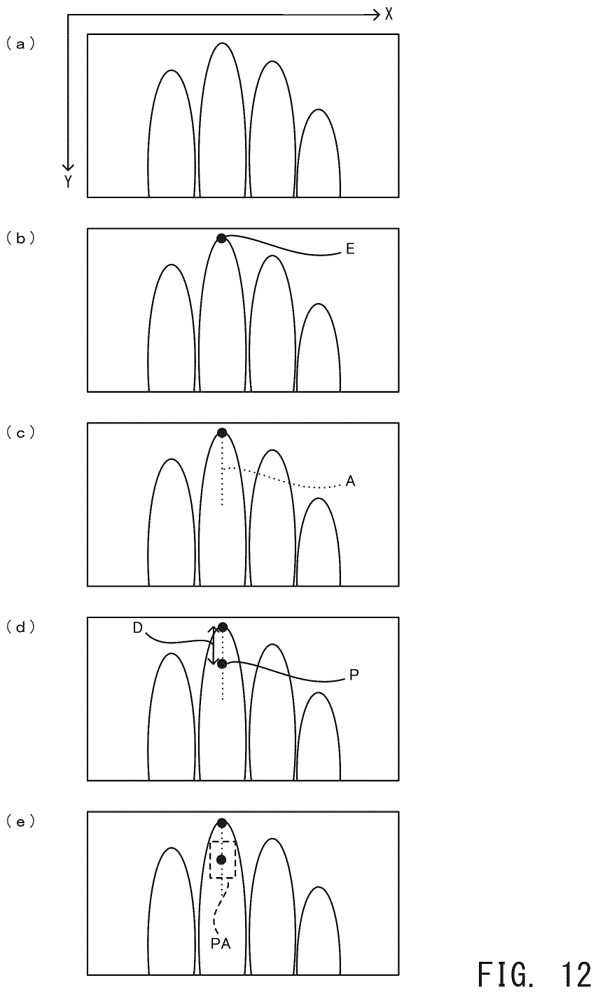

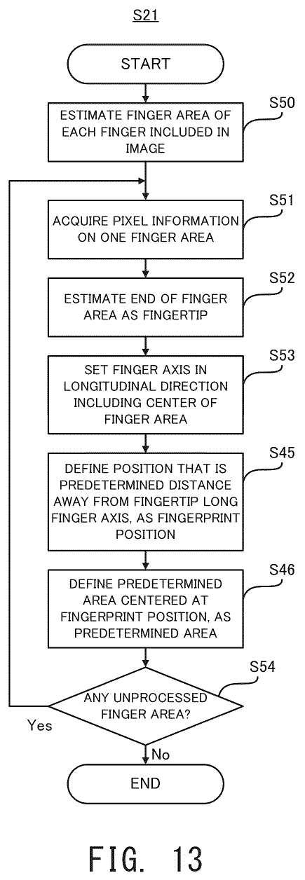

With reference to FIG. 11 and FIG. 12, a flow of a fingerprint area determination operation performed by the optical coherence tomography image generation apparatus 4 in the fourth example embodiment will be described. FIG. 11 is a flowchart illustrating the flow of the fingerprint area determination operation performed by the optical coherence tomography image generation apparatus 4 in the fourth example embodiment. FIG. 12 is a conceptual diagram of the fingerprint area determination operation performed by the optical coherence tomography image generation apparatus 4 in the fourth example embodiment.

In the fourth example embodiment, the target of the optical coherence tomography image generation is a hand. In the fourth example embodiment, the determination unit 212 determines the fingerprint area of at least one of the fingers of the hand, as at least one scan area, on the basis of the stereoscopic 3D image SI illustrated in FIG. 12(a), for example. In the fourth example embodiment, the determination unit 212 may estimate the fingertip of at least one of the fingers of the hand on the basis of the stereoscopic 3D image SI, may estimate a finger axis, and may determine the fingerprint area including an area that is a predetermined distance away from the fingertip along the finger axis, as at least one of the plurality of scan areas. The flowchart illustrated in FIG. 11 may illustrate a detailed operation flow of the operation in the step S21 in FIG. 4(a).

As illustrated in FIG. 11, the determination unit 212 sums pixel values of pixels arranged in the Y direction, for each X position in the X direction (step S40). The determination unit 212 may sum luminance values of pixels arranged in the Y direction, for each X position in the X direction. The X direction may be a horizontal direction in the case illustrated in FIG. 3 (b), for example. In this case, the Y direction may be a vertical direction in the case illustrated in FIG. 3 (b). The optical coherence tomography image generation apparatus 4 may guide the direction of the finger held over the camera unit 110 such that the longitudinal direction of the finger is the Y direction. In a case where the direction of the finger held over the camera unit 110 is determined, one direction in the stereoscopic 3D image SI may be estimated to be the finger axis.

Alternatively, the X direction may coincide with a moving direction of the irradiation position of the light by the scanner unit 210 in the B scan described above (also referred to as the “scanning line direction” and the “fast axis direction of the scanning”). Furthermore, the Y direction may be a direction perpendicular to the X direction, and may coincide with the “slow axis direction of the scanning” described above.

The determination unit 212 extracts at least one peak X position where the sum of the pixel values of the pixels arranged in the Y-direction is at the peak (step S41). The determination unit 212 may detect as many peaks as the number of the fingers included in the stereoscopic 3D image SI. In many cases, the sum of the pixel values of the pixels arranged in the Y-direction is at the peak, at the X position where there is the fingertip. Therefore, the determination unit 212 may estimate the X position where the sum of the pixel values of the pixels arranged in the Y-direction is at the peak, as the X position where there is the fingertip.

The determination unit 212 calculates a Y-direction derivative, at the one peak X position (step S42). The determination unit 212 estimates a Y position in the Y-direction indicating a limiting value of the derivative calculated in step S42, as the fingertip (step S43). The determination unit 212 may obtain a change in the pixel values in the Y direction, and may estimate that the fingertip is at a Y position with a large change.

That is, the determination unit 212 may estimate a part where there is a sharp change in a position in a lateral direction of at least one of the fingers of the hand and there is a sudden change in a position in a longitudinal direction of the finger, as the fingertip, on the basis of the stereoscopic 3D image SI. The determination unit 212 may estimate a fingertip E, as illustrated in FIG. 12(b), for example.

The determination unit 212 sets the finger axis along the Y direction from the estimated fingertip (step S44). The determination unit 212 may estimate an axis in the longitudinal direction of the finger including the center of a part with higher pixel values than those of surroundings, as the finger axis of the finger, on the basis of the stereoscopic 3D image SI. The determination unit 212 may estimate a finger axis A, as illustrated in FIG. 12(c), for example.

The determination unit 212 defines a position that is a predetermined distance away from the estimated fingertip along the set finger axis, as a fingerprint center position P (step S45). The determination unit 212 may define a position that is a predetermined distance D away from the fingertip E, as the fingerprint center position P, as illustrated in FIG. 12(d), for example. Instead of the predetermined distance D, the determination unit 212 may define a position that is a predetermined number of pixels away from the fingertip E, as the fingerprint center position P.

The determination unit 212 defines a predetermined area centered at the fingerprint center position P, as a fingerprint area PA (step S46). The determination unit 212 may define a predetermined rectangular area centered at the fingerprint center position P, as the fingerprint area PA, for example, as illustrated in FIG. 12(e). The determination unit 212 determines the fingerprint area PA including an area that is a predetermined distance away from a fingertip along the finger axis, as at least one of the plurality of scan areas.

The determination unit 212 determines whether or not there is an unprocessed peak position of the extracted peak positions (step S47). When there is an unprocessed peak position of the extracted peak positions (the step S47: Yes), the operation proceeds to the step S42. When there is no unprocessed peak position of the extracted peak positions (the step S47: No), the operation of determining the fingerprint area PA is ended.

The determination unit 212 may calculate the fingertip E, the finger axis A, the fingerprint center position P, and the fingerprint area PA for each of the images that constitute the stereoscopic 3D image SI, and may determine the three-dimensional position of the scan area, on the basis of the fingertip E, the finger axis A, the fingerprint center position P, and the fingerprint area PA in each image.

### [4-2: Technical Effect of Optical Coherence Tomography Image Generation Apparatus 4]

The optical coherence tomography image generation apparatus 4 in the fourth example embodiment is capable of easily and accurately determining the fingerprint area PA by estimating the fingertip and the finger axis. Since the optical coherence tomography image generation apparatus 4 estimates the fingertip in accordance with the pixel value, it is possible to easily and accurately determine the fingerprint area. Furthermore, since the optical coherence tomography image generation apparatus 4 estimates the finger axis in accordance with the pixel value, it is possible to easily and accurately determine the fingerprint area.

### 5: FIFTH EXAMPLE EMBODIMENT

An optical coherence tomography image generation apparatus, an optical coherence tomography image generation method, and a recording medium according to a fifth example embodiment will be described. The following describes the optical coherence tomography image generation apparatus, the optical coherence tomography image generation method, and the recording medium according to the fifth example embodiment, by using an optical coherence tomography image generation apparatus 5 to which the optical coherence tomography image generation apparatus, the optical coherence tomography image generation method, and the recording medium according to the fifth example embodiment are applied.

The optical coherence tomography image generation apparatus 5 in the fifth example embodiment is different in the area determination operation by the determination unit 212, from the optical coherence tomography image generation apparatus 4 in the fourth example embodiment. Other features of the optical coherence tomography image generation apparatus 5 may be the same as those of the optical coherence tomography image generation apparatus 4.

### [5-1: Fingerprint Area Determination Operation Performed By Optical Coherence Tomography Image Generation Apparatus 5]

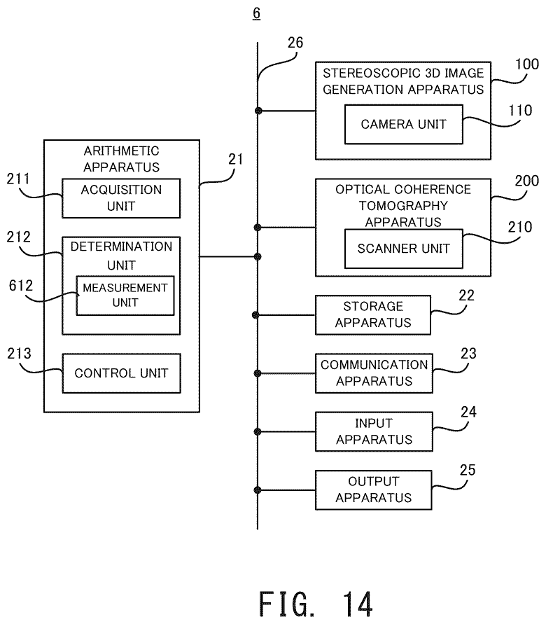

With reference to FIG. 13, a flow of the fingerprint area determination operation performed by the optical coherence tomography image generation apparatus 5 in the fifth example embodiment will be described. FIG. 13 is a flowchart illustrating the flow of the fingerprint area determination operation performed by the optical coherence tomography image generation apparatus 5 in the fifth example embodiment. FIG. 13 is a conceptual diagram illustrating the optical coherence tomography image generation operation performed by the optical coherence tomography image generation apparatus 5 in the fifth example embodiment.

In the fifth example embodiment, the target of the optical coherence tomography image generation is a hand. In the fifth example embodiment, the determination unit 212 determines the fingerprint area of at least one of the fingers of the hand, as at least one scan area, on the basis of the stereoscopic 3D image SI. In the fifth example embodiment, the determination unit 212 estimates the fingertip of at least one finger of the hand on the basis of the stereoscopic 3D image SI, estimates the finger axis, and determines the fingerprint area including an area that is a predetermined distance away from the fingertip along the finger axis, as at least one of the plurality of scan areas. The flowchart illustrated in FIG. 13 may illustrate a detailed operation flow of the operation in the step S21 in FIG. 4(a).

As illustrated in FIG. 13, the determination unit 212 estimates a finger area of each finger included in the image, on the basis of the stereoscopic 3D image SI (step S50). In the step S50, the determination unit 212 may perform Morphological (morphology) transformation, for example. Alternatively, in the step S50, the determination unit 212 may use MASK R-CNN that allows object detection and image segmentation, for example. According to the MASK R-CNN, even if there is an overlap between areas of targets, it is possible to separate it as different targets. Furthermore, according to the MASK R-CNN, even if there is an overlap between areas of targets, it is possible to obtain a boundary of the targets.

The determination unit 212 acquires pixel information on one finger area (step S51). In the step S51, the determination unit 212 may acquire the pixel data on the finger area, for example, by a Watershed algorithm. The determination unit 212 estimates an end of the finger area as the fingertip (step S52). The determination unit 212 may estimate a part where there is a sharp change in a position in a lateral direction of the finger area and there is a sudden change in a position in a longitudinal direction of the finger area, as the fingertip, on the basis of the pixel information.

The determination unit 212 sets the finger axis of the finger in the longitudinal direction including the center of the finger area (step S53). The determination unit 212 may estimate an axis in the longitudinal direction of the finger including the center of the finger area, as the finger axis of the finger. Alternatively, the determination unit 212 may estimate an intersection between the boundary of the finger area and the finger axis, as the fingertip, on the basis of the pixel information.

The determination unit 212 defines a position that is a predetermined distance away from the estimated fingertip along the set finger axis, as the fingerprint center position P (step S45). The determination unit 212 defines a predetermined area centered at the fingerprint center position P, as the fingerprint area PA (step S46). The determination unit 212 may determine the fingerprint area including an area that is a predetermined distance away from the estimated fingertip along the finger axis, as at least one of the plurality of scan areas.

The determination unit 212 determines whether or not there is an unprocessed finger area of the estimated finger areas (step S54). When there is an unprocessed finger area of the estimated finger areas (the step S54: Yes), the operation proceeds to the step S51. When there is no unprocessed finger area of the estimated finger areas (the step S54: No), the fingerprint area determination operation is ended.

### [5-2: Technical Effect of Optical Coherence Tomography Image Generation Apparatus 5]

Since the optical coherence tomography image generation apparatus 5 in the fifth example embodiment estimates the axis including the center of the finger area, as the finger axis, it is possible to easily and accurately determine the fingerprint area.

### 6: SIXTH EXAMPLE EMBODIMENT

An optical coherence tomography image generation apparatus, an optical coherence tomography image generation method, and a recording medium according to a sixth example embodiment will be described. The following describes the optical coherence tomography image generation apparatus, the optical coherence tomography image generation method, and the recording medium according to the sixth example embodiment, by using an optical coherence tomography image generation apparatus 6 to which the optical coherence tomography image generation apparatus, the optical coherence tomography image generation method, and the recording medium according to the sixth example embodiment are applied.

### [6-1: Configuration of Optical Coherence Tomography Image Generation Apparatus 6]

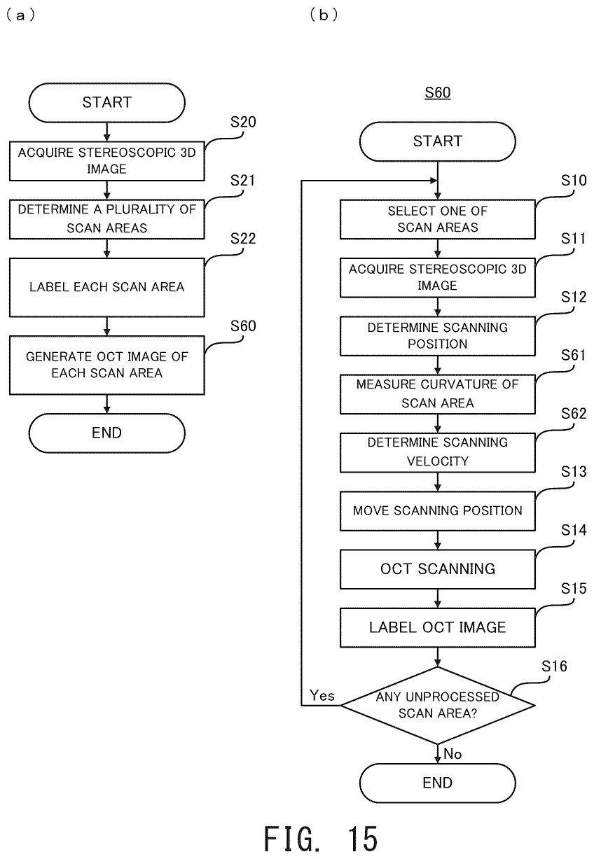

With reference to FIG. 14, a configuration of the optical coherence tomography image generation apparatus 6 in the sixth example embodiment will be described. FIG. 14 is a block diagram illustrating the configuration of the optical coherence tomography image generation apparatus 6 in the sixth example embodiment.

As illustrated in FIG. 14, the optical coherence tomography image generation apparatus 6 in the sixth example embodiment includes the arithmetic apparatus 21 and the storage apparatus 22, as in at least one of the optical coherence tomography image generation apparatus 2 in the second example embodiment to the optical coherence tomography image generation apparatus 5 in the fifth example embodiment. Furthermore, the optical coherence tomography image generation apparatus 6 includes the communication apparatus 23, the input apparatus 24, and the output apparatus 25, as in at least one of the optical coherence tomography image generation apparatus 2 in the second example embodiment to the optical coherence tomography image generation apparatus 5 in the fifth example embodiment. The optical coherence tomography image generation apparatus 6, however, may not include at least one of the communication apparatus 23, the input apparatus 24, and the output apparatus 25. The optical coherence tomography image generation apparatus 6 in the sixth example embodiment is different from at least one of the optical coherence tomography image generation apparatus 2 in the second example embodiment to the optical coherence tomography image generation apparatus 5 in the fifth example embodiment, in that the determination unit 212 provided in the arithmetic apparatus 21 includes a measurement unit 612. The measurement unit 612 measures curvatures of each of the plurality of scan areas, on the basis of the stereoscopic 3D image SI. Other features of the optical coherence tomography image generation apparatus 6 may be the same as those of at least one of the optical coherence tomography image generation apparatus 2 in the second example embodiment to the optical coherence tomography image generation apparatus 5 in the fifth example embodiment.

### [6-2: Optical Coherence Tomography Image Generation Operation Performed By Optical Coherence Tomography Image Generation Apparatus 6]

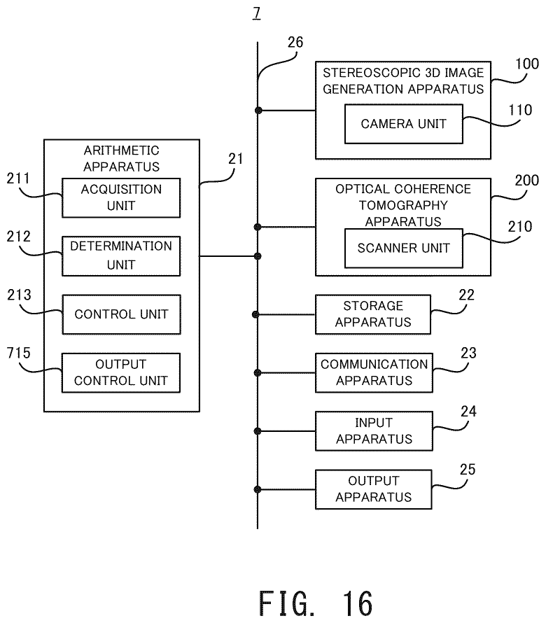

With reference to FIG. 15, a flow of the optical coherence tomography image generation operation performed by the optical coherence tomography image generation apparatus 6 in the sixth example embodiment will be described. FIG. 15 is a flowchart illustrating the flow of the optical coherence tomography image generation operation performed by the optical coherence tomography image generation apparatus 6 in the sixth example embodiment.

As illustrated in FIG. 15(a), the acquisition unit 211 acquires the stereoscopic 3D image SI of the target (step S20). The determination unit 212 determines the plurality of scan areas on the target, on the basis of the stereoscopic 3D image SI of the hand (step S21). For example, as illustrated in FIG. 10, the determination unit 212 may determine the four divided areas obtained by dividing the rectangular area including the fingerprint in the entire area to the first joint from the fingertip into four pieces, as the plurality of scan areas.

The determination unit 212 labels each of the plurality of scan areas (step S22). For example, as illustrated in FIG. 10, the determination unit 212 may label a lower left fingerprint area as “1.” In addition, the determination unit 212 may label a lower right fingerprint area as “2.” In addition, the determination unit 212 may label an upper left fingerprint area as “3.” In addition, the determination unit 212 may label an upper right fingerprint area as “4.”

The control unit 213 generates the optical coherence tomography image of each scan area (step S60). The operation in the step S60 is illustrated in FIG. 15(b). As illustrated in FIG. 15(b), the control unit 213 selects one of the plurality of scan areas (step S10). The acquisition unit 211 acquires the stereoscopic 3D image SI of the selected one scan area (step S11). As in the second example embodiment and the third example embodiment, however, the control unit 213 may not acquire the stereoscopic 3D image SI of the selected one scan area.

The determination unit 212 determines the OCT scanning position corresponding to the selected one scan area, on the basis of the stereoscopic 3D image SI (step S12). The measurement unit 612 measures the curvature of the selected one scan area, on the basis of the stereoscopic 3D image SI (step S61). The control unit 213 determines a scanning velocity for the selected one scan area, on the basis of the curvature (step S62). As the scanning velocity, the control unit 213 may determine a moving velocity of the irradiation position of the light in the selected one scan area.

The control unit 213 moves the scanner unit 210 to the OCT scanning position corresponding to the one scan area (step S13). For example, in the case illustrated in FIG. 10, the control unit 213 may move the scanner unit 210 to the OCT scanning position corresponding to selected one of the fingerprint areas 1, 2, 3, and 4.

The control unit 213 relatively moves the irradiation position of light for capturing the optical coherence tomography image of the one scan area with respect to the one scan area, at the scanning velocity determined in the step S62, and controls the scanning by the light of the one scan area (step S14). The control unit 213 may control the OCT scan by the scanner unit 210.

The determination unit 212 performs the same labeling as that of the scan area, on the captured optical coherence tomography image of the one scan area (step S15).

The determination unit 212 determines whether or not there is a scan area in which the step S10 to the step S15 are not yet performed (step S16). When there is a scan area in which the step S10 to the step S15 are not yet performed (the step S16: Yes), the operation proceeds to the step S10. When there is no scan area in which the step S10 to the step S15 are not yet performed (the step S16: No), the optical coherence tomography image generation operation in the sixth example embodiment is ended. The determination unit 212 may repeat the step S10 to the step S15 as many times as the number of divisions of the area.

For example, in the case illustrated in FIG. 10, in many cases, the curvatures of the fingerprint areas 3 and 4 are greater than those of the fingerprint areas 1 and 2. In other words, the surface of the fingerprint area is inclined to a plane perpendicular to the optical axis of the light emitted to the fingerprint area in many cases. Since the fingerprint area with a large curvature, or the fingerprint area with a large inclination to the plane perpendicular to the optical axis, tends to provide low resolution, it is preferable to obtain information finely/in more detail. In a case where the target is scanned at a low scanning velocity, it is possible to acquire information on the target at fine spacing, as compared with a case where the target is scanned at a high scanning velocity. Therefore, for example, the control unit 213 may relatively move the fingerprint areas 3 and 4 at a lower scanning velocity than one for the fingerprint areas 1 and 2.

### [6-3: Technical Effect of Optical Coherence Tomography Image Generation Apparatus 6]

Since the optical coherence tomography image generation apparatus 6 in the sixth example embodiment changes the scanning velocity for relatively moving the irradiation position of the light in accordance with the curvature, it is possible to generate the high-accuracy optical coherence tomography image.

### 7: SEVENTH EXAMPLE EMBODIMENT

An optical coherence tomography image generation apparatus, an optical coherence tomography image generation method, and a recording medium according to a seventh example embodiment will be described. The following describes the optical coherence tomography image generation apparatus, the optical coherence tomography image generation method, and the recording medium according to the seventh example embodiment, by using an optical coherence tomography image generation apparatus 7 to which the optical coherence tomography image generation apparatus, the optical coherence tomography image generation method, and the recording medium according to the seventh example embodiment are applied.

### [7-1: Configuration of Optical Coherence Tomography Image Generation Apparatus 7]

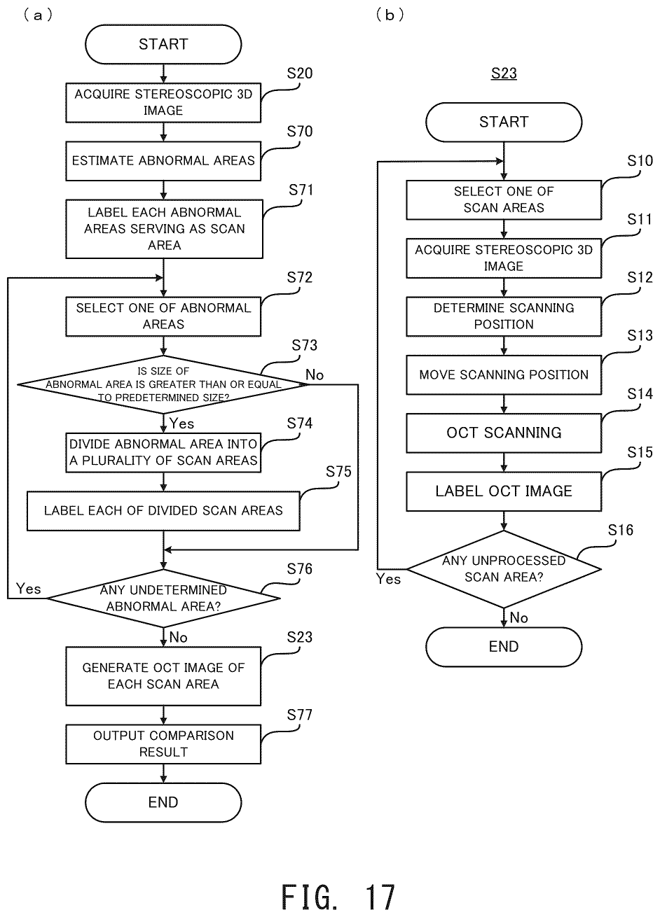

With reference to FIG. 16, a configuration of the optical coherence tomography image generation apparatus 7 in the seventh example embodiment will be described. FIG. 16 is a block diagram illustrating the configuration of the optical coherence tomography image generation apparatus 7 in the seventh example embodiment.

As illustrated in FIG. 16, the optical coherence tomography image generation apparatus 7 in the seventh example embodiment includes the arithmetic apparatus 21 and the storage apparatus 22, as in at least one of the optical coherence tomography image generation apparatus 2 in the second example embodiment to the optical coherence tomography image generation apparatus 6 in the sixth example embodiment. Furthermore, the optical coherence tomography image generation apparatus 7 may include the communication apparatus 23, the input apparatus 24, and the output apparatus 25, as in at least one of the optical coherence tomography image generation apparatus 2 in the second example embodiment to the optical coherence tomography image generation apparatus 6 in the sixth example embodiment. The optical coherence tomography image generation apparatus 7, however, may not include at least one of the communication apparatus 23, the input apparatus 24, and the output apparatus 25. The optical coherence tomography image generation apparatus 7 in the seventh example embodiment is different from at least one of the optical coherence tomography image generation apparatus 2 in the second example embodiment to the optical coherence tomography image generation apparatus 6 in the sixth example embodiment, in that the target is a skin and the arithmetic apparatus 21 includes an output control unit 715. Other features of the optical coherence tomography image generation apparatus 7 may be the same as those of at least one of the optical coherence tomography image generation apparatus 2 in the second example embodiment to the optical coherence tomography image generation apparatus 6 in the sixth example embodiment.

### [7-2: Operation by Optical Coherence Tomography Image Generation Apparatus 7]

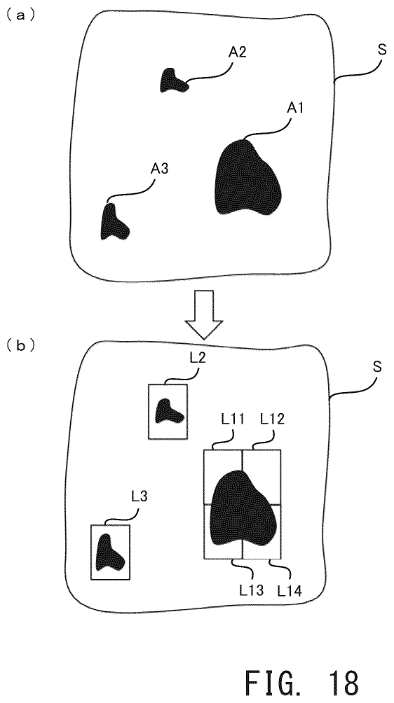

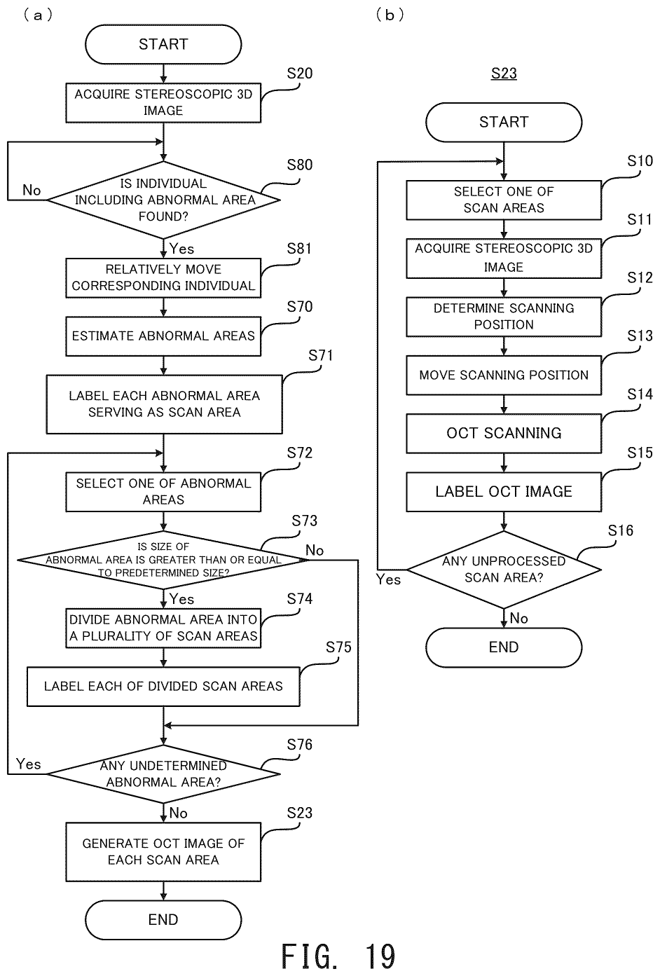

With reference to FIG. 17 and FIG. 18, a flow of the optical coherence tomography image generation operation performed by the optical coherence tomography apparatus 7 in the seventh example embodiment will be described. FIG. 17 is a flowchart illustrating the flow of the optical coherence tomography image generation operation performed by the optical coherence tomography image generation apparatus 7 in the seventh example embodiment. FIG. 18 is a conceptual diagram illustrating the scan area determination operation performed by the optical coherence tomography image generation apparatus 7 in the seventh example embodiment.

As illustrated in FIG. 17(a), the acquisition unit 211 acquires the stereoscopic 3D image SI of the skin serving as the target (step S20).

In a case where there are parts where a state of the skin is different to a predetermined amount or more from those in adjacent parts, the determination unit 212 estimates the corresponding parts as abnormal areas, and determines the abnormal areas as the plurality of scan areas, on the basis of the stereoscopic 3D image SI of the skin (step S70). For example, as illustrated in FIG. 18(a), the determination unit 212 may estimates parts A1, A2, and A3 where the state of the skin S is different to a predetermined amount or more from those in adjacent parts, as the abnormal area, and may determine the abnormal areas as the plurality of scan areas. As described in the fourth example embodiment, the determination unit 212 may estimate the abnormal areas by using a method of estimating points with a significant change in the pixel values in the stereoscopic 3D image SI as the boundary of the area. Furthermore, as described in the fifth example embodiment, the determination unit 212 may divide the stereoscopic 3D image SI by using the method of image segmentation and may estimate the abnormal areas.

The determination unit 212 labels each of the plurality of abnormal areas (step S71). For example, in the case illustrated in FIG. 18(a), the determination unit 212 may label the abnormal area A1 as “L1.” The determination unit 212 may label the abnormal area A2 as “L2.” The determination unit 212 may label the abnormal area A3 as “L3.”

The determination unit 212 selects one of the plurality of abnormal areas (step S72). The determination unit 212 determines whether or not a size of the selected one abnormal area is greater than or equal to a predetermined size (step S73). When the size of the selected one abnormal area is less than the predetermined size (the step S73: No), the operation proceeds to a step S76. When the size of the selected one abnormal area is greater than or equal to the predetermined size (the step S73: Yes), the determination unit 212 divides the abnormal area into a plurality of scan areas (step S74). For example, as illustrated in FIG. 18(b), the determination unit 212 may divide the abnormal area A1 labeled as “L1” into four pieces.

The determination unit 212 labels each of the plurality of divided scan areas (step S75). For example, as illustrated in FIG. 18(b), the determination unit 212 may label a first quarter scan area as “L11”, may label a second quarter scan area as “L12”, may label a third quarter scan area as “L13”, and may label a fourth quarter scan area as “L14”.

The determination unit 212 determines whether or not there is an abnormal area in which the step S72 to the step S75 are not yet performed (step S76). When there is an abnormal area in which the step S72 to the step S75 are not yet performed (the step S76: Yes), the operation proceeds to the step S72. When there is no abnormal area in which the step S72 to the step S75 are not yet performed (the step S76: No), the control unit 213 generates the optical coherence tomography image of each scan area (step S23).

The operation of the step S23 is illustrated in FIG. 17(b). As illustrated in FIG. 17(b), the control unit 213 selects one of the plurality of abnormality areas (step S10).

The acquisition unit 211 acquires the stereoscopic 3D image SI of the selected one abnormal area (step S11). As the second, third, and sixth example embodiments, however, the control unit 213 may not acquire the stereoscopic 3D image SI of the selected one abnormal area.

The determination unit 212 determines the OCT scanning position corresponding to the selected one abnormal area, on the basis of the stereoscopic 3D image SI (step S12). The control unit 213 moves the scanner unit 210 to the OCT scanning position corresponding to the one abnormal area (step S13). The control unit 213 relatively moves the irradiation position of light for capturing the optical coherence tomography image of the one abnormal area with respect to the one abnormal area, and controls the scanning by the light of the one abnormal area (step S14). The determination unit 212 performs the same labeling as that of the abnormal area, on the captured optical coherence tomography image of the one abnormal area (step S15).

The determination unit 212 determines whether or not there is an abnormal area in which the step S10 to the step S15 are not yet performed (step S16). When there is an abnormal area in which the step S10 to the step S15 are not yet performed (the step S16: Yes), the operation proceeds to the step S10. When there is no abnormal area in which the step S10 to the step S15 are not yet performed (the step S16: No), the operation proceeds to a step S77.

The output control unit 715 outputs a comparison result between optical coherence tomography results of the abnormal area on the skin acquired in different timing (step S77). The output control unit 715 may add at least one of information indicating a positional relation between the abnormal area and skin features (wrinkles, irregularities, bones, skin thickness, moles, stains, and edges) and information indicating a positional relation between the plurality of abnormal areas, to each of the abnormal areas. The output control unit 715 may compare the abnormal area with the position stored and the scanned abnormal area when the imaging is performed again at a later date, and may output information indicating a change in size of an internal pathology/lesion or the like. By comparing the optical coherence tomography images, it is possible to properly grasp a change in the state of an inside of the skin over time.

### [7-3: Technical Effect of Optical Coherence Tomography Image Generation Apparatus 7]

The optical coherence tomography image generation apparatus 7 in the seventh example embodiment is capable of detecting the state of an inside of the part where the state of the skin is different to a predetermined amount or more from those in adjacent parts, accurately and in a non-invasive manner.

### 8: EIGHTH EXAMPLE EMBODIMENT

An optical coherence tomography image generation apparatus, an optical coherence tomography image generation method, and a recording medium according to an eighth example embodiment will be described. The following describes the optical coherence tomography image generation apparatus, the optical coherence tomography image generation method, and the recording medium according to the eighth example embodiment, by using an optical coherence tomography image generation apparatus 8 to which the optical coherence tomography image generation apparatus, the optical coherence tomography image generation method, and the recording medium according to the eighth example embodiment are applied.

The optical coherence tomography image generation apparatus 8 in the eighth example embodiment is different from the optical coherence tomography image generation apparatus 7 in the seventh example embodiment, in that the target is an agricultural crop. Other features of the optical coherence tomography image generation apparatus 8 may be the same as at least one of those of the optical coherence tomography image generation apparatus 7.

### [8-1: Operation by Optical Coherence Tomography Image Generation Apparatus 8]

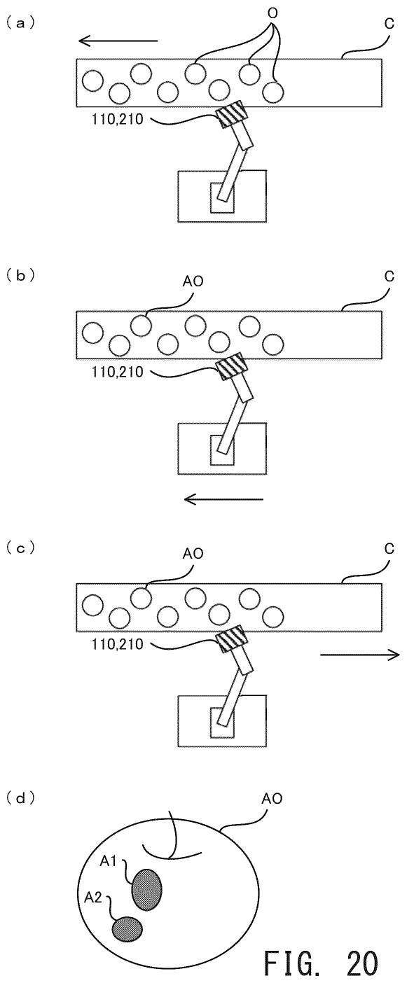

With reference to FIG. 19 and FIG. 20, a flow of the optical coherence tomography image generation operation performed by the optical coherence tomography apparatus 8 performs in the eighth example embodiment will be described. FIG. 19 is a flowchart illustrating the flow of the optical coherence tomography image generation operation performed by the optical coherence tomography image generation apparatus 8 in the eighth example embodiment. FIG. 20 is a conceptual diagram illustrating the scan area determination operation performed by the optical coherence tomography image generation apparatus 8 in the eighth example embodiment.

As illustrated in FIG. 19(a), the acquisition unit 211 acquires the stereoscopic 3D image SI of the agricultural crop serving as the target (step S20). The acquisition unit 211 may acquire the stereoscopic 3D image SI including a plurality of agricultural crops as the target. For example, as illustrated in FIG. 20(a), a plurality of agricultural crops O may be placed on a conveyor C. By operation of the conveyor C, the plurality of agricultural crops O may be relatively moved with respect to the camera unit 110 and the scanner unit 210 (in the case illustrated in FIG. 20(a), may be moved from right to left in the drawing).

The determination unit 212 determines whether or not an individual AO including the abnormal areas is found from the target included in the stereoscopic 3D image SI (step S80). When the individual AO including the abnormal areas is not found (the step S80: No), the operation proceeds to the step S80.

When the individual AO including the abnormal areas is found (the step S80: Yes), the control unit 213 relatively moves the corresponding individual AO, the camera unit 110, and the scanner unit 210 (step S81). As illustrated in FIG. 20(b), when the individual AO including the abnormal areas is found, the control unit 213 may stop the operation of the conveyor C, and may move the camera unit 110 and the scanner unit 210 to a position where the corresponding individual AO can be imaged. Alternatively, as illustrated in FIG. 20(c), the control unit 213 controls a moving direction of the conveyor C reversely, and may move the corresponding individual AO to a position where it can be imaged by the camera unit 110 and the scanner unit 210.

The determination unit 212 identifies the abnormal areas in the corresponding individual AO (step S70). In a case where there are parts where a state of a surface of the agricultural croup is different to a predetermined amount or more from those in adjacent parts, the determination unit 212 may estimate the corresponding parts as the abnormal areas, on the basis of the stereoscopic 3D image SI of the skin. For example, as illustrated in FIG. 20(d), the determination unit 212 may estimate parts A1 and A2 where the state of the surface of the agricultural croup is different to a predetermined amount or more from those in adjacent parts, as the abnormal area, and may determine the abnormal areas as the plurality of scanned areas. The determination unit 212 may estimate the abnormal areas by using the same method as at least one of those in the fourth, fifth, and seventh example embodiments.

The determination unit 212 labels each of the plurality of abnormal areas (step S71). For example, in the case illustrated in FIG. 20(d), the determination unit 212 may label the abnormal area A1 as “L1.” The determination unit 212 may label the abnormal area A2 as “L2.”

The determination unit 212 selects one of the plurality of abnormal areas (step S72). The determination unit 212 determines whether or not the size of the selected one abnormal area is greater than or equal to a predetermined size (step S73). When the size of the selected one abnormal area is less than the predetermined size (the step S73: No), the operation proceeds to the step S76. When the size of the selected one abnormal area is greater than or equal to the predetermined size (the step S73: Yes), the determination unit 212 divides the abnormal area into a plurality of scan areas (step S74). The determination unit 212 labels each of the plurality of divided scan areas (step S75).

The determination unit 212 determines whether or not there is an abnormal area in which the step S72 to the step S75 are not yet performed (step S76). When there is an abnormal area in which the step S72 to the step S75 are not yet performed (the step S76: Yes), the operation proceeds to the step S72. When there is no abnormal area in which the step S72 to the step S75 are not yet performed (the step S76: No), the control unit 213 generates the optical coherence tomography image of each scan area (step S23).

The operation of the step S23 is illustrated in FIG. 19(b). As illustrated in FIG. 19(b), the control unit 213 selects one of the plurality of abnormality areas (step S10).

The acquisition unit 211 acquires the stereoscopic 3D image SI of the selected one abnormal area (step S11). As the second, third, sixth, and seventh example embodiments, however, the control unit 213 may not acquire the stereoscopic 3D image SI of the selected one abnormal area.

The determination unit 212 determines the OCT scanning position corresponding to the selected one abnormal area, on the basis of the stereoscopic 3D image SI (step S12). The control unit 213 moves the scanner unit 210 to the OCT scanning position corresponding to the one abnormal area (step S13). The control unit 213 relatively moves the irradiation position of the light for capturing the optical coherence tomography image of the one abnormal area with respect to the one abnormal area, and controls the scanning by the light of the one abnormal area (step S14). The determination unit 212 performs the same labeling as that of the abnormal area, on the captured optical coherence tomography image of the one abnormal area (step S15).

The determination unit 212 determines whether or not there is an abnormal area in which the step S10 to the step S15 are not yet performed (step S16). When there is an abnormal area in which the step S10 to the step S15 are not yet performed (the step S16: Yes), the operation proceeds to the step S10. When there is no abnormal area in which the step S10 to the step S15 are not yet performed (the step S16: No), the operation of generating the optical coherence tomography image of the abnormal area is ended.

In the example embodiments other than the eighth example embodiment, as illustrated in FIG. 20(c), the scan area may be moved to the optical coherence tomography scanning position by moving the target.

### [8-2: Technical Effect of Optical Coherence Tomography Image Generation Apparatus 8]

The optical coherence tomography image generation apparatus 8 in the eighth example embodiment is capable of detecting the state of an inside of the part where the state of the surface is different to a predetermined amount or more from those in adjacent parts, accurately and in a non-invasive manner. It is possible to determine whether the part where or not the state of the surface is different to a predetermined amount or more from those in adjacent parts, is caused by a crop disease or the like, accurately and in a non-invasive manner.

### 9: NINTH EXAMPLE EMBODIMENT

An optical coherence tomography image generation apparatus, an optical coherence tomography image generation method, and a recording medium according to a ninth example embodiment will be described. The following describes the optical coherence tomography image generation apparatus, the optical coherence tomography image generation method, and the recording medium according to the ninth example embodiment, by using an optical coherence tomography image generation apparatus 9 to which the optical coherence tomography image generation apparatus, the optical coherence tomography image generation method, and the recording medium according to the ninth example embodiment are applied.

The optical coherence tomography image generation apparatus 9 in the ninth example embodiment is different in the control operation by the control unit 213, from the optical coherence tomography image generation apparatus 2 in the second example embodiment to the optical coherence tomography image generation apparatus 8 in the eighth example embodiment. Other features of the optical coherence tomography image generation apparatus 9 may be the same as those of at least one of the optical coherence tomography image generation apparatus 2 to the optical coherence tomography image generation apparatus 8.

In the ninth example embodiment, the controller 213 has a degree of freedom in at least one of translation and rotation, has a degree of freedom in at least one of translation and rotation to control at least one of the irradiation position and an irradiation angle of the light with respect to the target, and controls at least one of the irradiation position and the irradiation angle of the light with respect to the target.

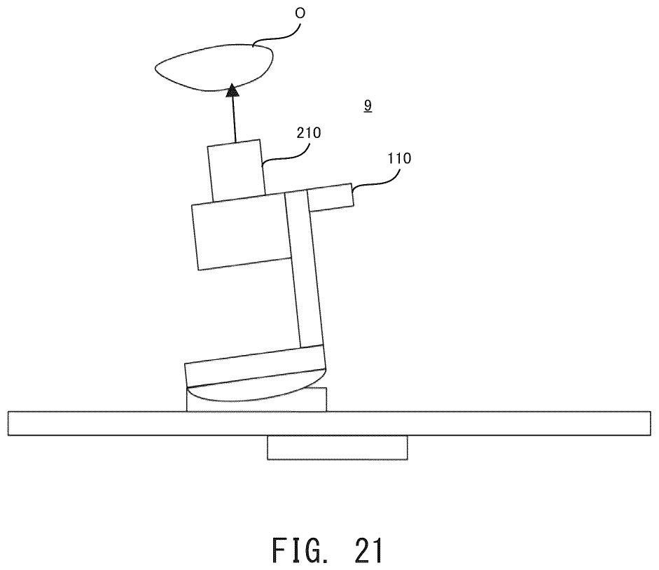

For example, as illustrated in FIG. 21, when light is applied at an angle of 90 degrees to the target O, it is possible to acquire a higher-accuracy optical coherence tomography image. In the ninth example embodiment, the control unit 213 may perform a control of driving the camera unit 110 and the scanner unit 210 in an X-axis direction, a Y-axis direction, a yaw-direction, a roll direction, and a pitch direction.

As the curvature of the target, which is a curved surface, with respect to the camera-portion 110 and the scanner unit 210 increases, the imaging accuracy of the target, which is a curved surface, may not be satisfactorily maintained in some cases. In the case of imaging the target, which is a curved surface, if the camera unit 110 and the scanner unit 210 is driven only in the X-axis direction and the Y-axis direction, the imaging accuracy of the curved surface may not be maintained as satisfactorily as at an end of the target. Therefore, in the ninth example embodiment, the camera unit 110 and the scanner unit 210 are driven not only in the X-axis direction and the Y-axis direction, but also in the yaw direction, the roll direction, and the pitch direction. By this, the optical coherence tomography image generation apparatus 9 is capable of generating the optical coherence tomography image of the target, which is a curved surface, more accurately.

The control unit 213 may associate a control value of the scanner unit 210 in the optical coherence tomography, with each optical coherence tomography image. The control value of the scanner unit 210 may be, for example, an angle in the yaw direction, an angle in the roll direction, and an angle in the pitch direction.

The optical coherence tomography image generation apparatus 9 may calculate a difference in angles between adjacent scan areas and may perform a correction for reducing an angle deviation when composing the plurality of optical coherence tomography images. Since the scanner unit 210 performs the optical coherence tomography in a non-contact state, the target may move when capturing the optical coherence tomography image. In contrast, each of an inclination in the yaw direction, an inclination in the roll direction, and an inclination in the pitch direction may be calculated on the basis of the stereoscopic 3D image SI at the time of the optical coherence tomography scanning of each scan area, as to how much the target moves from an initial value in the optical coherence tomography scanning of each scan area, and the respective inclinations may be reflected in the correction at the time of composition.

In the ninth example embodiment, the camera unit 110 and the scanner unit 210 may not be an integral apparatus. On the other hand, the control values of five axes are required as parameters of coordinate transformation between the camera unit 110 and the scanner unit 210.

### [Technical Effect of Optical Coherence Tomography Image Generation Apparatus 9]

Since the optical coherence tomography image generation apparatus 9 in the ninth example embodiment controls at least one of the irradiation position and the irradiation angle of the light with a degree of freedom in at least one of translation and rotation, it is possible to determine the optical axis direction in accordance with the angle of the surface of the target, and it is possible to generate the accurate optical coherence tomography image.

### 10: TENTH EXAMPLE EMBODIMENT

An optical coherence tomography image generation apparatus, an optical coherence tomography image generation method, and a recording medium according to a tenth example embodiment will be described. The following describes the optical coherence tomography image generation apparatus, the optical coherence tomography image generation method, and the recording medium according to the tenth example embodiment, by using an optical coherence tomography image generation apparatus 10 to which the optical coherence tomography image generation apparatus, the optical coherence tomography image generation method, and the recording medium according to the tenth example embodiment are applied.

The optical coherence tomography image generation apparatus 10 in the tenth example embodiment is different from the optical coherence tomography image generation apparatus 9 in the ninth example embodiment, in that the degree of freedom in translation and rotation is 6. Other features of the optical coherence tomography image generation apparatus 10 may be the same as at least one of those of the optical coherence tomography image generation apparatus 9.

In the tenth example embodiment, the control unit 213 may perform a control of driving the camera unit 110 and the scanner unit 210 in the X-axis direction, the Y-axis direction, a Z-axis direction, the yaw-direction, the roll direction, and the pitch direction. That is, the control unit 213 may perform the control in the Z-axis direction, in addition to the X-axis direction, the Y-axis direction, the yaw-direction, the roll direction, and the pitch direction.

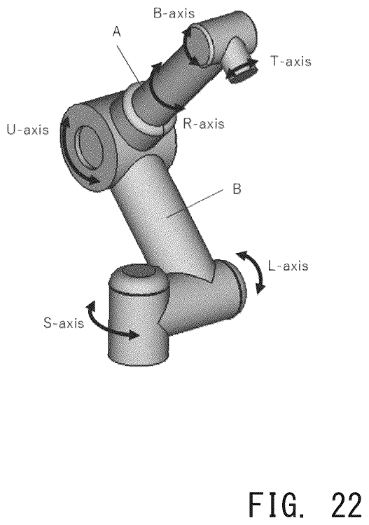

As illustrated in FIG. 22, the scanner unit 210 may be fixed to an end of an arm A. In FIG. 22, an S-axis may indicate rotation in a horizontal plane, an L-axis may indicate back and forth movement, a U-axis may indicate raising and lowering an arm, a R-axis may indicate rotation of the arm, a B-axis may indicate raising and lowering the end of the arm, and a T-axis may indicate rotation of the end of the arm.

Since the optical coherence tomography image generation apparatus 10 in the tenth example embodiment further has the degree of freedom in the Z-axis direction and controls at least one of the irradiation position and the irradiation angle of the light, it is possible to set a distance between the surface of the target and the scanner unit to an optimum distance for the optical coherence tomography scanning, and it is possible to generate the higher-accuracy optical coherence tomography image.

### 11: ELEVENTH EXAMPLE EMBODIMENT

An optical coherence tomography image generation apparatus, an optical coherence tomography image generation method, and a recording medium according to an eleventh example embodiment will be described. The following describes the optical coherence tomography image generation apparatus, the optical coherence tomography image generation method, and the recording medium according to the eleventh example embodiment, by using an optical coherence tomography image generation apparatus 11 to which the optical coherence tomography image generation apparatus, the optical coherence tomography image generation method, and the recording medium according to the eleventh example embodiment are applied.

### [11-1: Configuration of Optical Coherence Tomography Image Generation Apparatus 11]

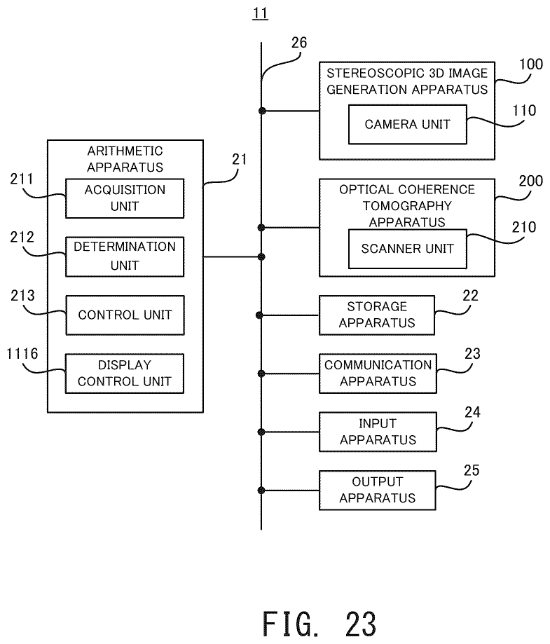

With reference to FIG. 23, a configuration of the optical coherence tomography image generation apparatus 11 in the eleventh example embodiment will be described. FIG. 23 is a block diagram illustrating the configuration of the optical coherence tomography image generation apparatus 11 in the eleventh example embodiment.

As illustrated in FIG. 23, the optical coherence tomography image generation apparatus 11 in the eleventh example embodiment is different from the optical coherence tomography image generation apparatus 2 in the second example embodiment to the optical coherence tomography image generation apparatus 11 in the tenth example embodiment, in that the arithmetic apparatus 21 includes a display control unit 1116. Other features of the optical coherence tomography image generation apparatus 11 may be the same as those of at least one of the optical coherence tomography apparatus 2 to the optical coherence tomography image generation apparatus 10.

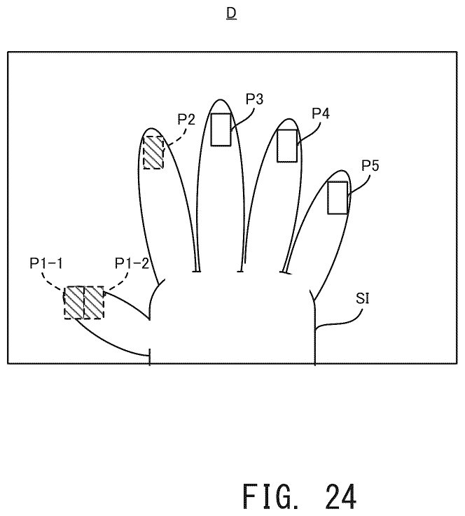

FIG. 24 illustrates a management screen D displayed by the display control unit 1116. The display control unit 1116 may allow the output apparatus 25 serving as a display, to display the management screen D as illustrated in FIG. 24. The management screen D may be a screen browsed by an operator of the optical coherence tomography image generation apparatus 11.

The display control unit 1116 superimposes and displays information indicating the plurality of scan areas, on the stereoscopic 3D image SI. In the case illustrated in FIG. 24, the display control unit 1116 superimposes and displays rectangular frames P1-1, P1-2, P2, P3, P4, and P5 indicating the plurality of scan areas determined by the determination unit 212, on the stereoscopic 3D image SI. The scan area corresponding to the first finger is greater than or equal to a predetermined size, and may be divided into two scan areas P1-1 and P1-2.

In the case illustrated in FIG. 24, the control unit 213 may generate the optical coherence tomography image in order of P1-1, P1-2, P2, P3, P4, and P5. For the scan area that is already imaged, a solid line frame may be changed to a broken line frame, and the generated optical coherence tomography image may be superimposed. The display control unit 1116 may further superimpose and display the optical coherence tomography image captured by the control unit 213, on areas corresponding to the plurality of scan areas in the stereoscopic 3D image SI. In the case illustrated in FIG. 24, the control unit 213 may have already generated the optical coherence tomography image for the scan areas P1-1, P1-2, and P2.

The display control unit 1116 may change a line type of the frame indicating the scan area and may superimpose and display the optical coherence tomography image in a case where the optical coherence tomography image has a predetermined or higher quality. For example, in a case where there is still a scan area with a frame indicating unprocessed after all the scan areas are processed, the operator may guide a target person of the imaging by the optical coherence tomography image generation apparatus 11, to image again the finger that is not scanned.

### [11-2: Technical Effect of Optical Coherence Tomography Image Generation Apparatus 11]

The optical coherence tomography image generation apparatus 11 in the eleventh example embodiment allows the operator of the optical coherence tomographic apparatus 11 to easily grasp a situation of the optical coherence tomography image generation by visual recognition.

### 12: TWELFTH EXAMPLE EMBODIMENT

An optical coherence tomography image generation apparatus, an optical coherence tomography image generation method, and a recording medium according to a twelfth example embodiment will be described. The following describes the optical coherence tomography image generation apparatus, the optical coherence tomography image generation method, and the recording medium according to the twelfth example embodiment, by using an optical coherence tomography image generation apparatus 12 to which the optical coherence tomography image generation apparatus, the optical coherence tomography image generation method, and the recording medium according to the twelfth example embodiment are applied.

### [12-1: Configuration of Optical Coherence Tomography Image Generation Apparatus 12]

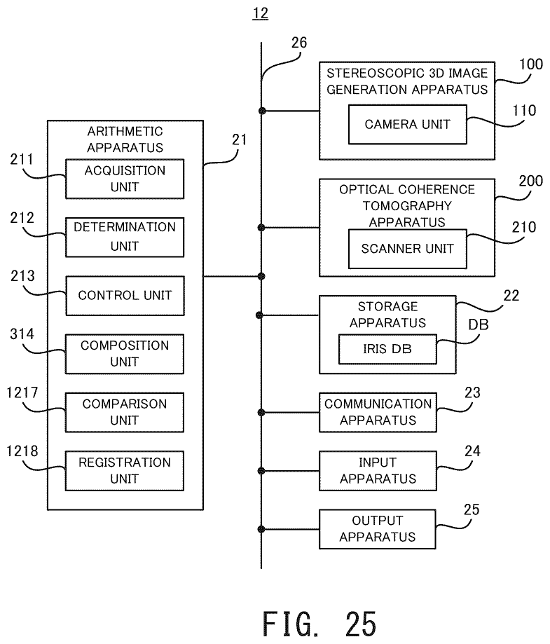

With reference to FIG. 25, a configuration of the optical coherence tomography image generation apparatus 12 in the twelfth example embodiment will be described. FIG. 25 is a block diagram illustrating the configuration of the optical coherence tomography image generation apparatus 12 in the twelfth example embodiment.

As illustrated in FIG. 25, the optical coherence tomography image generation apparatus 12 in the twelfth example embodiment is different from the optical coherence tomography image generation apparatus 2 in the second example embodiment to the optical coherence tomography image generation apparatus 12 in the eleventh example embodiment, in that the target is an iris, that the arithmetic apparatus 21 includes a comparison unit 1217 and a registration unit 1218, and that the storage apparatus 22 stores an iris database DB in which registration iris images are registered. The storage apparatus 22, however, may not store the iris database DB. In the twelfth example embodiment, the camera unit 110 may be an infrared camera that captures an infrared image. Other features of the optical coherence tomography image generation apparatus 12 may be the same as those of at least one of the optical coherence tomography apparatus 2 to the optical coherence tomography image generation apparatus 11.

### [12-2: Operation by Optical Coherence Tomography Image Generation Apparatus 12]

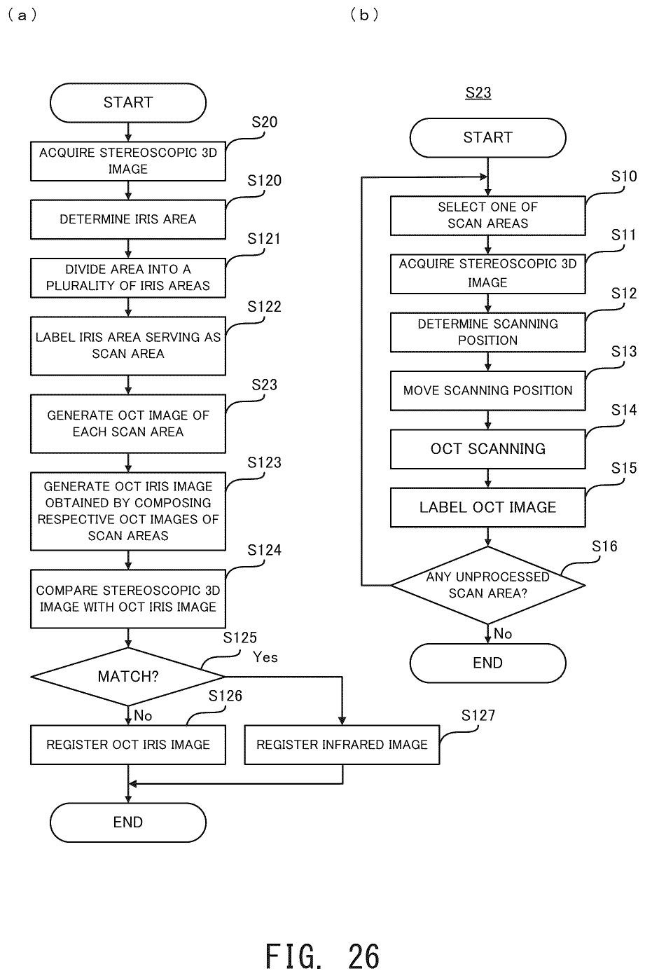

With reference to FIG. 26, a flow of the optical coherence tomography image generation operation performed by the optical coherence tomography apparatus 12 in the twelfth example embodiment will be described. FIG. 26 is a flowchart illustrating the flow of the optical coherence tomography image generation operation performed by the optical coherence tomography image generation apparatus 12 in the twelfth example embodiment.

As illustrated in FIG. 26(a), the acquisition unit 211 acquires the stereoscopic 3D image SI of the iris serving as the target (step S20). The determination unit 212 determines an iris area serving as an imaging area, on the basis of the stereoscopic 3D image SI (step S120). The determination unit 212 may determine the iris area with a size larger than an image size obtained by one C-scan, on the basis of the stereoscopic 3D image SI.

The determination unit 212 divides the iris area and determines a plurality of scan areas (step S121). The determination unit 212 labels each of the plurality of scan areas (step S122).

The control unit 213 generates the optical coherence tomography image of each scan area (step S23). The operation of the step S23 is illustrated in FIG. 26(b). As illustrated in FIG. 26(b), the control unit 213 selects one of the plurality of scan areas (step S10). The acquisition unit 211 acquires the stereoscopic 3D image SI of the selected one scan area (step S11). As in at least one of the second, third, sixth, and eighth example embodiments, however, the control unit 213 may not acquire the stereoscopic 3D image SI of the selected one scan area. The determination unit 212 determines the OCT scanning position corresponding to the selected one scan area, on the basis of the stereoscopic 3D image SI (step S12). The control unit 213 moves the scanner unit 210 to the OCT scanning position corresponding to the one scan area (step S13). The control unit 213 relatively moves the irradiation position of the light for capturing the optical coherence tomography image of the one scan area with respect to the one scan area, and controls the scanning by the light of the one scan area (step S14). The determination unit 212 performs the same labeling as that of the scan area, on the captured optical coherence tomography image of the one scan area (step S15).

The determination unit 212 determines whether or not there is a scan area in which the step S10 to the step S15 are not yet performed (step S16). When there is a scan area in which the step S10 to the step S15 are not yet performed (the step S16: Yes), the operation proceeds to the step S10. When there is no scan area in which the step S10 to the step S15 are not yet performed (the step S16: No), the operation proceeds to a step S123.

The composition unit 314 generates an optical coherence tomography iris image obtained by composing the respective optical coherence tomography images of the scan areas (step S123).

The comparison unit 1217 compares the stereoscopic 3D image SI of the corresponding iris with the optical coherence tomography iris image of the corresponding iris generated by the control unit 213 (step S124). The comparison unit 1217 determines whether the stereoscopic 3D image SI of the corresponding iris matches the optical coherence tomography iris image of the corresponding iris (step S125).

When the stereoscopic 3D image SI of the corresponding iris does not match the optical coherence tomography iris image of the corresponding iris (the step S125: No), the comparison unit 1217 registers the optical coherence tomography iris image of the corresponding iris in the iris database DB, as a registration image for iris recognition/authentication (step S126). When the stereoscopic 3D image SI of the corresponding iris matches the optical coherence tomography iris image of the corresponding iris (the step S125: Yes), the comparison unit 1217 registers at least one of the images that constitute the stereoscopic 3D image SI of the corresponding iris in the iris database DB, as the registration image for iris recognition (step S127).

The case where the stereoscopic 3D image SI of the corresponding iris does not match the optical coherence tomography iris image of the corresponding iris captured by the control unit 213, may be a case where a target person wears colored contact lenses. In such a case, the infrared image cannot be used as the registration image that is a matching target. Thus, instead of the infrared image, the optical coherence tomography image may be registered in the iris database DB as the registration image that is a matching target.

### [12-3: Technical Effect of Optical Coherence Tomography Image Generation Apparatus 12]

For example, in a case where the target person of the iris recognition wears the colored contact lenses, it is hard to acquire features of the iris of the target person from an image in which a surface of an eyeball is imaged. In this instance, since the optical coherence tomography image generation apparatus 12 in the twelfth example embodiment is allowed to use the optical coherence tomography image of the iris, it is possible to acquire the features of the iris.

### 13: SUPPLEMENTARY NOTES

With respect to the example embodiment described above, the following Supplementary Notes are further disclosed.

### Supplementary Note 1

An optical coherence tomography image generation apparatus including:
an acquisition unit that acquires a stereoscopic 3D image of a target;
a determination unit that determines a plurality of scan areas on the target, on the basis of the stereoscopic 3D image; and
a control unit that relatively moves an irradiation position of light for capturing an optical coherence tomography image of the target with respect to the target, and that controls scanning by the light of each of the plurality of scan areas.

### Supplementary Note 2

The optical coherence tomography image generation apparatus according to Supplementary Note 1, wherein the determination unit determines an imaging area on the target on the basis of the stereoscopic 3D image, and divides the imaging area to determine the plurality of scan areas.

### Supplementary Note 3

The optical coherence tomography image generation apparatus according to Supplementary Note 1 or 2, wherein
the determination unit includes a measurement unit that measures curvatures of the plurality of scan areas, on the basis of the stereoscopic 3D image, and
the control unit changes a velocity of relatively moving the irradiation position of the light on the basis of the curvatures.

### Supplementary Note 4

The optical coherence tomography image generation apparatus according to any one of Supplementary Notes 1 to 3, wherein
the target is a hand, and
the determination unit determines a fingerprint area of each of two or more of fingers of the hand, as the plurality of scan areas, on the basis of the stereoscopic 3D image.

### Supplementary Note 5

The optical coherence tomography image generation apparatus according to any one of Supplementary Notes 1 to 4, wherein
the target is a hand, and
the determination unit estimates a fingertip of at least one of fingers of the hand, on the basis of the stereoscopic 3D image, and determines a fingerprint area including at least a part of an area extending to a first joint on the finger from the fingertip toward a base of the finger, as at least one of the plurality of scan areas.

### Supplementary Note 6

The optical coherence tomography image generation apparatus according to any one of Supplementary Notes 1 to 5, wherein
the target is a hand, and
the determination unit estimates a fingertip of at least one of fingers of the hand, on the basis of the stereoscopic 3D image, estimates a finger axis of the finger, and determines a fingerprint area including an area that is a predetermined distance away from the fingertip along the finger axis, as at least one of the plurality of scan areas.

### Supplementary Note 7

The optical coherence tomography image generation apparatus according to any one of Supplementary Notes 1 to 6, wherein
the target is a hand, and
the determination unit estimates a part where there is a sharp change in a position in a lateral direction of at least one of fingers of the hand and there is a sudden change in a position in a longitudinal direction of the finger, as a fingertip, on the basis of the stereoscopic 3D image.

### Supplementary Note 8

The optical coherence tomography image generation apparatus according to any one of Supplementary Notes 5 to 7, wherein the determination unit estimates an axis in a longitudinal direction of the finger including a center of a part with higher pixel values than those of surroundings, as a finger axis of the finger, on the basis of the stereoscopic 3D image, and determines a fingerprint area including an area that is a predetermined distance away from the fingertip along the finger axis, as at least one of the plurality of scan areas.

### Supplementary Note 9

The optical coherence tomography image generation apparatus according to any one of Supplementary Notes 5 to 7, wherein the determination unit estimates a finger area of at least one of fingers of the hand, on the basis of the stereoscopic 3D image, estimates an axis in a longitudinal direction of the finger including a center of the finger area, as a finger axis of the finger, and determines a fingerprint area including an area that is a predetermined distance away from the fingertip along the finger axis, as at least one of the plurality of scan areas.

### Supplementary Note 10

The optical coherence tomography image generation apparatus according to any one of Supplementary Notes 1 to 3, wherein
the target is a skin, and
in a case where there are parts where a state of the skin is different to a predetermined amount or more from those in adjacent parts, the determination unit estimates the corresponding parts as abnormal areas, and determines the abnormal areas as the plurality of scan areas, on the basis of the stereoscopic 3D image.

### Supplementary Note 11

The optical coherence tomography image generation apparatus according to any one of Supplementary Notes 1 to 10, wherein the control unit has a degree of freedom of at least one of translation and rotation, and controls at least one of the irradiation position and an irradiation angle of the light with respect to the target.

### Supplementary Note 12

The optical coherence tomography image generation apparatus according to any one of Supplementary Notes 1 to 11, further including a display unit that superimposes and displays information indicating the plurality of scan areas on the stereoscopic 3D image, wherein the display unit further superimposes and displays an optical coherence tomography image captured by the control unit, on area corresponding to the plurality of scan areas in the stereoscopic 3D image.

### Supplementary Note 13

The optical coherence tomography image generation apparatus according to any one of Supplementary Notes 1 to 3, 11, and 12, wherein
the target is an iris, and
the optical coherence tomography image generation apparatus further comprises, in a case where the stereoscopic 3D image of the iris does not match an optical coherence tomography image of the iris captured by the control unit, a registration unit that registers the optical coherence tomography image of the corresponding iris, as a registration image for iris recognition.

### Supplementary Note 14

An optical coherence tomography image generation method including:
acquiring a stereoscopic 3D image of a target;
determining a plurality of scan areas on the target, on the basis of the stereoscopic 3D image; and
relatively moving an irradiation position of light for capturing an optical coherence tomography image of the target with respect to the target, and controlling scanning by the light of each of the plurality of scan areas.

### Supplementary Note 15

A recording medium on which a computer program that allows a computer to execute an optical coherence tomography image generation method is recorded, the optical coherence tomography image generation method including:
acquiring a stereoscopic 3D image of a target;
determining a plurality of scan areas on the target, on the basis of the stereoscopic 3D image; and
relatively moving an irradiation position of light for capturing an optical coherence tomography image of the target with respect to the target, and controlling scanning by the light of each of the plurality of scan areas.

At least a part of the constituent components of each of the example embodiments described above can be combined with at least another part of the constituent components of each of the example embodiments described above, as appropriate. A part of the constituent components of each of the example embodiments described above may not be used. Furthermore, to the extent permitted by law, all the references (e.g., publications) cited in this disclosure are incorporated by reference as a part of the description of this disclosure.

This disclosure is not limited to the examples described above and is allowed to be changed, if desired, without departing from the essence or spirit of this disclosure which can be read from the claims and the entire identification. An optical coherence tomography image generation apparatus, an optical coherence tomography image generation method, and a recording medium with such changes are also intended to be within the technical scope of this disclosure.

### DESCRIPTION OF REFERENCE CODES

1, 2, 3, 4, 5, 6, 7, 8, 9, 10, 11, 12 The optical coherence tomography image generation apparatus
100 Stereoscopic 3D image generation apparatus
110 Camera unit
200 The optical coherence tomography apparatus
210 Scanner unit
11, 211 Acquisition unit
12, 212 Determination unit
13, 213 Control unit
314 Composition unit
612 Measurement unit
715 Output control unit
1116 Display control unit
1217 Comparison unit
1218 Registration unit
SI Stereoscopic 3D image
E Finger tip
A Finger axis
P Fingerprint center position

What is claimed is:

1. An optical coherence tomography image generation apparatus comprising:
at least one memory that is configured to store instructions; and
at least one first processor that is configured to execute the instructions to:
acquire a stereoscopic 3D image of a target;
determine a plurality of scan areas on the target, on the basis of the stereoscopic 3D image; and
relatively move an irradiation position of light for capturing an optical coherence tomography image of the target with respect to the target, and control scanning by the light of each of the plurality of scan areas.

2. The optical coherence tomography image generation apparatus according to claim 1, wherein the at least one first processor is configured to execute the instructions to determine an imaging area on the target on the basis of the stereoscopic 3D image, and divide the imaging area to determine the plurality of scan areas.

3. The optical coherence tomography image generation apparatus according to claim 1, wherein
the at least one first processor is configured to execute the instructions to
measure curvatures of the plurality of scan areas, on the basis of the stereoscopic 3D image, and
change a velocity of relatively moving the irradiation position of the light on the basis of the curvatures.

4. The optical coherence tomography image generation apparatus according to claim 1, wherein
the target is a hand, and
the at least one first processor is configured to execute the instructions to determine a fingerprint area of each of two or more of fingers of the hand, as the plurality of scan areas, on the basis of the stereoscopic 3D image.

5. The optical coherence tomography image generation apparatus according to claim 1, wherein
the target is a hand, and
the at least one first processor is configured to execute the instructions to estimate a fingertip of at least one of fingers of the hand, on the basis of the stereoscopic 3D image, and determine a fingerprint area including at least a part of an area extending to a first joint on the finger from the fingertip toward a base of the finger, as at least one of the plurality of scan areas.

6. The optical coherence tomography image generation apparatus according to claim 1, wherein
the target is a hand, and
the at least one first processor is configured to execute the instructions to estimate a fingertip of at least one of fingers of the hand, on the basis of the stereoscopic 3D image, estimate a finger axis of the finger, and determine a fingerprint area including an area that is a predetermined distance away from the fingertip along the finger axis, as at least one of the plurality of scan areas.

7. The optical coherence tomography image generation apparatus according to claim 1, wherein
the target is a hand, and
the at least one first processor is configured to execute the instructions to estimate a part where there is a sharp change in a position in a lateral direction of at least one of fingers of the hand and there is a sudden change in a position in a longitudinal direction of the finger, as a fingertip, on the basis of the stereoscopic 3D image.

8. The optical coherence tomography image generation apparatus according to claim 5, wherein the at least one first processor is configured to execute the instructions to estimate an axis in a longitudinal direction of the finger including a center of a part with higher pixel values than those of surroundings, as a finger axis of the finger, on the basis of the stereoscopic 3D image, and determine a fingerprint area including an area that is a predetermined distance away from the fingertip along the finger axis, as at least one of the plurality of scan areas.

9. The optical coherence tomography image generation apparatus according to claim 5, wherein the at least one first processor is configured to execute the instructions to estimate a finger area of at least one of fingers of the hand, on the basis of the stereoscopic 3D image, estimate an axis in a longitudinal direction of the finger including a center of the finger area, as a finger axis of the finger, and determine a fingerprint area including an area that is a predetermined distance away from the fingertip along the finger axis, as at least one of the plurality of scan areas.

10. The optical coherence tomography image generation apparatus according to claim 1, wherein
the target is a skin, and
in a case where there are parts where a state of the skin is different to a predetermined amount or more from those in adjacent parts, the at least one first processor is configured to execute the instructions to estimate the corresponding parts as abnormal areas, and determine the abnormal areas as the plurality of scan areas, on the basis of the stereoscopic 3D image.

11. The optical coherence tomography image generation apparatus according to claim 1, wherein the at least one first processor is configured to execute the instructions to have a degree of freedom of at least one of translation and rotation, and control at least one of the irradiation position and an irradiation angle of the light with respect to the target.

12. The optical coherence tomography image generation apparatus according to claim 1, wherein the at least one first processor is configured to execute the instructions to
superimpose and display information indicating the plurality of scan areas on the stereoscopic 3D image, and
further superimpose and display an optical coherence tomography image captured, on area corresponding to the plurality of scan areas in the stereoscopic 3D image.

13. The optical coherence tomography image generation apparatus according to claim 1, wherein
the target is an iris, and
the at least one first processor is configured to execute the instructions to, in a case where the stereoscopic 3D image of the iris does not match an optical coherence tomography image of the iris captured, register the optical coherence tomography image of the corresponding iris, as a registration image for iris recognition.

14. An optical coherence tomography image generation method comprising:
acquiring a stereoscopic 3D image of a target;
determining a plurality of scan areas on the target, on the basis of the stereoscopic 3D image; and
relatively moving an irradiation position of light for capturing an optical coherence tomography image of the target with respect to the target, and controlling scanning by the light of each of the plurality of scan areas.

15. A non-transitory recording medium on which a computer program that allows a computer to execute an optical coherence tomography image generation method is recorded, the optical coherence tomography image generation method including:
acquiring a stereoscopic 3D image of a target;
determining a plurality of scan areas on the target, on the basis of the stereoscopic 3D image; and
relatively moving an irradiation position of light for capturing an optical coherence tomography image of the target with respect to the target, and controlling scanning by the light of each of the plurality of scan areas.
